(19) United States
(12) Patent Application Publication
(10) Pub. No.: US 2026/0317708 A1
(43) Pub. Date: Oct. 15, 2026

(54) INTERFERON-ENHANCED CAR-T CELLS

(71) Applicant: THE UNIVERSITY OF CHICAGO, Chicago, IL (US)

(72) Inventors: Jun Huang, Chicago, IL (US); Yifei Hu, Chicago, IL (US); Erting Tang, Chicago, IL (US)

(21) Appl. No.: 19/648,883

(22) Filed: Apr. 15, 2026

Related U.S. Application Data
(60) Provisional application No. 63/789,043, filed on Apr. 15, 2025.

(51) Int. Cl. C12N 5/0783; A61K 40/11; A61K 40/31; A61K 40/42; A61P 35/00; C07K 16/28

(52) U.S. Cl. CPC C12N 5/0636; A61K 40/11; A61K 40/31; A61K 40/4211; A61P 35/00; C07K 16/2803; C12N 2501/24; C12N 2510/00

(57) ABSTRACT

This disclosure relates to a population of interferon (IFN)-enhanced chimeric antigen receptor (CAR)-T cells and methods of their production. The methods include treating a population of CAR-T cells expressing a CAR gene construct with a concentration of an IFN receptor agonist sufficient to produce a population of IFN-enhanced CAR-T cells, wherein compared to an untreated control CAR-T cell population, the population of IFN-enhanced CAR-T cells exhibits increased expression of one or more interferon-stimulated genes. The disclosure also relates to a pharmaceutical composition comprising the population of IFN-enhanced chimeric antigen receptor (CAR)-T cells and methods of treating cancer with the pharmaceutical composition.

### CROSS-REFERENCE TO RELATED APPLICATIONS

This application claims priority to U.S. provisional application No. 63/789,043, filed Apr. 15, 2025, the disclosure of which is expressly incorporated by reference herein.

### STATEMENT REGARDING FEDERALLY SPONSORED RESEARCH

This invention was made with government support under AI144245 and AI169159 awarded by the National Institutes of Health. The government has certain rights in the invention.

### BACKGROUND OF DISCLOSURE

Chimeric antigen receptor (CAR)-T cell therapy has advanced the standard-of-care for cancers such as hematologic malignancies whose complete response rates can range from 39-71% across relapsed/refractory diffuse large B-cell lymphoma (r/r DLBCL) (Neelapu et al., 2017, N. Engl. J. Med 377, 2531-2544) acute lymphoblastic leukemia (Maude et al., 2018, N. Engl. J. Med 378, 439-448), and multiple myeloma (Rodriguez-Otero et al., 2023, Engl. J. Med 388, 1002-1014; San-Miguel et al., 2023, N. Engl. J. Med 389, 335-347). However, the therapy's effectiveness is constrained by significant rates of therapy failure. For instance, in r/r DLBCL, only ~40% of patients exhibit a complete response, whereas ~20% of patients exhibit progressive disease, as defined by the International Working Group response criteria (Neelapu et al., 2017, N. Engl. J. Med 377, 2531-2544). Patients who cannot clear their lymphoma burden have limited therapeutic options and often succumb to their disease. Existing studies have correlated progressive disease with diminished CAR T-cell polyfunctionality (Rossi et al., Blood 132, 804-814), increased T-cell exhaustion signatures within CAR T-cell products (Fraietta et al., 2018, Nat. Med 24, 563-571; Deng et al., 2020, Nat. Med 26, 1878-1887; Finney et al., 2019, J. Clin. Invest. 129, 2123-2132), inhibition by CAR Tregs (Good et al., 2022, Nat. Med 28, 1860-1871), as well as failure of CAR T cells to persist and expand in vivo (Neelapu et al., 2017, N. Engl. J. Med 377, 2531-2544; Schuster et al., 2019, N. Engl. J. Med 380, 45-56), among others (Cheng et al., 2019, Frontiers in Oncology 9, 1237).

Thus, there remains a need to improve the efficacy of CAR-T cell therapy in treating cancer.

### SUMMARY OF THE DISCLOSURE

This disclosure describes a population of interferon (IFN)-enhanced chimeric antigen receptor (CAR)-T cells and methods of their production. The disclosure also relates to a pharmaceutical composition comprising the population of IFN-enhanced CAR-T cells and methods of treating cancer with the pharmaceutical composition.

In the first aspect, the present disclosure provides a population of IFN-enhanced CAR-T cells having about 40% to about 70% maximal expression of one or more interferon-stimulated genes induced by treatment with an IFN receptor agonist

In one embodiment of the first aspect, the interferon-stimulated gene is one or more of OAS1/2/3, OASL, ADAR, IFITM1/2/3, IFI6, ITI16, ZAP, APOBEC3, ISG20, SOCS1, IRF7, MX1, and ISG15. In one embodiment of the first aspect, the interferon-stimulated gene is IRF7. In one embodiment of the first aspect, the IFN-enhanced CAR-T cells bind to CD19, CD20, CD22, or BCMA.

In one embodiment of any of the preceding embodiments, the IFN receptor agonist is an IFN type I (IFN-I) receptor agonist, an IFN type III (IFN-III) receptor agonist, or a combination thereof. In one embodiment, the IFN-I receptor agonist comprises IFN-α, IFN-β, IFN-ε, IFN-κ, IFN-ω, or a combination thereof. In one embodiment, the IFN-III receptor agonist comprises IFN-λ1, IFN-λ2, IFN-λ3, IFN-λ4, or a combination thereof.

In one embodiment of any of the preceding embodiments, the IFN-I receptor agonist is IFN-α2. In one embodiment, the concentration of the IFN-α2 is between about 100 IU/mL and about 1000 IU/mL. In one embodiment, the concentration of the IFN-α2 is about 300 IU/mL.

In one embodiment of any of the preceding embodiments, the population of IFN-enhanced CAR-T cells express a CAR gene construct that is an axicabtagene ciloleucel construct or a lisocabtagene maraleucel construct.

In one embodiment of any of the preceding embodiments, CAR-T cells are isolated from peripheral blood mononuclear cells (PBMCs) obtained from a donor prior to treatment with the IFN receptor agonist. In one embodiment of any of the preceding embodiments, the population of IFN-enhanced CAR-T cells produces more IL-2, TNF, and/or IFNγ upon stimulation compared to a control population of CAR-T cells that is produced without treatment with the IFN receptor agonist. In one embodiment of any of the preceding embodiments, the population of IFN-enhanced CAR-T cells expresses a higher level of interferon-stimulated genes compared to a control population of CAR-T cells that is produced without treatment with the IFN receptor agonist. In one embodiment of any of the preceding embodiments, the population of IFN-enhanced CAR-T cells undergoes the same rate of T cell activation-induced apoptosis as a control population of CAR-T cells that is produced without treatment with the IFN receptor agonist. In one embodiment of any of the preceding embodiments, the population of IFN-enhanced CAR-T cells is more effective in killing tumor cells compared to a control population of CAR-T cells that is produced without treatment with the IFN receptor agonist. In one embodiment of any of the preceding embodiments, the population of IFN-enhanced CAR-T cells has more durable efficacy compared to a control population of CAR-T cells that is produced without treatment with the IFN receptor agonist.

In a second aspect, the present disclosure provides a method of producing a population of interferon (IFN)-enhanced chimeric antigen receptor (CAR)-T cells, comprising:
a) introducing a CAR gene construct into a population of T cells to produce a population of CAR-T cells; and
b) co-culturing the population of CAR-T cells with a concentration of an IFN receptor agonist to obtain a population of IFN-enhanced CAR-T cells that exhibit about 40% to about 70% maximal expression of an interferon-stimulated gene.

In a third aspect, the present disclosure provides a method of preparing a CAR-T cell population, comprising:
treating a population of CAR-T cells expressing a CAR gene construct with an IFN receptor agonist to produce a population of IFN-enhanced CAR-T cells,
wherein compared to an untreated control CAR-T cell population, the population of IFN-enhanced CAR-T cells exhibits increased expression of one or more interferon-stimulated genes.

In one embodiment of any of the preceding methods described above, the method further comprises activating T cell receptor (TCR) signaling in the population of T cells or in the population of CAR-T cells before step (b). In one embodiment, the TCR signaling is activated by CD3 and/or CD28.

In one embodiment of any of the preceding methods described above, the method further comprises step (c) expanding the population of IFN-enhanced CAR-T cells.

In one embodiment of any of the preceding methods or embodiments thereof, the interferon-stimulated gene is OAS1/2/3, OASL, ADAR, IFITM1/2/3, IFI6, IFI16, ZAP, APOBEC3, ISG20, SOCS1, IRF7, MX1, and/or ISG15. In one embodiment of any of the preceding methods, the interferon-stimulated gene is IRF7. In one embodiment of any of the preceding methods or embodiments thereof, the CAR gene construct encodes a CAR that binds to CD19, CD20, CD22, or BCMA.

In one embodiment of any of the preceding methods or embodiments thereof, the IFN receptor agonist is an IFN type I (IFN-I) receptor agonist, an IFN type III (IFN-III) receptor agonist, or a combination thereof. In one embodiment, the IFN-I receptor agonist comprises IFN-α, IFN-β, IFN-ε, IFN-κ, IFN-ω, or a combination thereof. In one embodiment, the IFN-III receptor agonist comprises IFN-λ1, IFN-λ2, IFN-λ3, IFN-λ4, or a combination thereof.

In one embodiment of any of the preceding methods or embodiments thereof, the IFN-I receptor agonist is IFN-α2. In one embodiment, the concentration of the IFN-α2 is between about 100 IU/mL and about 1000 IU/mL. In one embodiment, the concentration of the IFN-α2 is about 300 IU/mL.

In one embodiment of any of the preceding methods or embodiments thereof, the CAR gene construct is an axicabtagene ciloleucel construct or a lisocabtagene maraleucel construct. In one embodiment, the CAR gene construct is introduced into T cells via a viral or a non-viral vector. In one embodiment, the viral vector comprises a lentivirus or a retrovirus. In one embodiment, the non-viral vector comprises a transposon, an mRNA molecule, a liposome, a molecular conjugate, and/or a lipid nanoparticle.

In one embodiment of any of the preceding methods or embodiments thereof, the T cells are isolated from peripheral blood mononuclear cells (PBMCs) obtained from a donor before step (a).

In one embodiment of any of the methods or embodiments thereof, the population of IFN-enhanced CAR-T cells produces more IL-2, TNF, and/or IFNγ upon stimulation compared to a control population of CAR-T cells that is produced without treatment with the IFN receptor agonist. In one embodiment of any of the preceding methods or embodiments thereof, the population of IFN-enhanced CAR-T cells expresses a higher level of interferon-stimulated genes compared to a control population of CAR-T cells that is produced without treatment with the IFN receptor agonist. In one embodiment of any of the preceding methods or embodiments thereof, the population of IFN-enhanced CAR-T cells undergoes the same rate of T cell activation-induced apoptosis as a control population of CAR-T cells that is produced without treatment with the IFN receptor agonist. In one embodiment of any of the methods or embodiments thereof, the population of IFN-enhanced CAR-T cells is more effective in killing tumor cells compared to a control population of CAR-T cells that is produced without treatment with the IFN receptor agonist. In one embodiment of any of the preceding In one embodiment of any of the methods or embodiments thereof, the population of IFN-enhanced CAR-T cells has more durable efficacy compared to a control population of CAR-T cells that is produced without treatment with the IFN receptor agonist.

In a fourth aspect, the present disclosure provides a pharmaceutical composition comprising the population of IFN signaling-enhanced CAR-T cells of the third aspect.

In a fifth aspect, the present disclosure provides a method of treating cancer in a patient, comprising administering a therapeutically effective amount of the pharmaceutical composition of the fourth aspect to the patient.

In one embodiment of the fifth aspect, the cancer is leukemia, multiple myeloma, diffuse large B cell lymphoma, acute myeloid leukemia, Mantle Cell lymphoma, follicular lymphoma, primary mediastinal B-cell lymphoma, acute lymphoblastic leukemia, or chronic lymphocytic leukemia. In one embodiment, the cancer is diffuse large B cell lymphoma.

In one embodiment of the fifth aspect, the population of IFN-enhanced CAR-T cells is produced from T cells that are isolated from PBMCs obtained from the patient or from a donor.

In the ninth aspect, the present disclosure provides a method of treating cancer in a subject in need thereof, comprising:
a) administering a therapeutically amount of a pharmaceutical composition comprising a population of IFN-I-enhanced CAR-T cells to the subject; and
b) increasing overall survival of the subject by at least about 2 days.

In one embodiment of the ninth aspect, the cancer is leukemia, multiple myeloma, diffuse large B cell lymphoma, acute myeloid leukemia, Mantle Cell lymphoma, follicular lymphoma, primary mediastinal B-cell lymphoma, acute lymphoblastic leukemia, or chronic lymphocytic leukemia. In one embodiment, the cancer is diffuse large B cell lymphoma.

In one embodiment of the ninth aspect, the method further comprising increasing progression-free survival, increasing radiographic response, and/or increasing patient quality of life.

In one embodiment of the ninth aspect, the population of IFN-enhanced CAR-T cells is produced from T cells that are isolated from PBMCs obtained from the subject or from a donor.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure and are incorporated in and constitute a part of this specification. The drawings illustrate one or more embodiment(s) of the disclosure, and together with the description, serve to explain the principles and operation of the disclosure.

FIG. 1A is a cartoon depicting a study design of single-cell analysis on infusion products of patients who received axi-cel from discovery and validation datasets. CR, complete responder, N-CR, non-complete responder.

FIG. 1B shows a UMAP plot demonstrating 7 T-cell clusters (proliferating, Naïve-activated, CD4⁺ Memory, CD4⁺ Treg, CD8⁺ central memory (CM), CD8⁺ effector memory (EM), and unconventional) in infusion products from the discovery dataset. No patient-specific or response-specific clusters were found.

FIG. 1C shows violin plots depicting normalized expression levels of representative markers for each cluster.

FIG. 1D shows stacked bar graphs depicting T-cell cluster proportions for each patient in the discovery dataset.

FIG. 1E shows UMAP plots demonstrating the distributions of the 7 T-cell clusters in each patient.

FIG. 1F shows boxplots depicting proportions of each phenotype within CR and N-CR T cells. Each dot represents a measurement from a single patient. Proportions in CR and N-CR T cells were compared by Wilcoxon rank-sum test. ns=no significant difference.

FIG. 1G shows a gene set enrichment analysis comparing CR and N-CR T cells. For each gene set, direction and statistical significance of enrichment are indicated by the circle's shade and size, respectively.

FIG. 1H shows a tilemap depicting scaled IFNα response signature scores in each T-cell cluster across patients.

FIG. 1I shows box plots depicting IFNα-response signature scores for CR and N-CR T cells within each T-cell cluster. Each dot represents one patient. Effect sizes were calculated using Cliff's δ, and statistical significance was assessed using the Wilcoxon rank-sum test.

FIG. 1J shows a UMAP plot demonstrating 7 T-cell clusters in infusion products from a validation dataset from Maus et al.

FIG. 1K shows violin plots depicting normalized expression levels of markers for each cluster.

FIG. 1L shows stacked bar graphs depicting T-cell cluster proportions for each patient in validation dataset.

FIG. 1M shows a volcano plot depicting differentially expressed genes (DEGs) between CR and N-CR across T cells. Genes involved in IFNα response gene set are dark gray. Dotted lines represent cutoff values for DEGs (log₂ fold-change of ±0.25) and significance threshold (adjusted p-value of 0.05).

FIG. 1N shows an enrichment plot for an IFNα response gene set differentially expressed between CR and N-CR T cells.

FIG. 1O shows a tilemap depicting scaled signature scores of IFNα response gene sets in each T-cell cluster across patients. No Tregs were found in P17 infusion products, marked with an X.

FIG. 1P shows box plots depicting IFNα-response signature scores for CR and N-CR T cells within each T-cell cluster. Each dot represents one patient. Effect sizes were calculated using Cliff's δ, and statistical significance was assessed using the Wilcoxon rank-sum test.

FIG. 2A shows a CD28-costimulated CAR (CD28-CAR) design based on the construct used in axicabtagene ciloleucel (“axi-cel”). TM: transmembrane; ICD: intracellular domain.

FIG. 2B shows a 4-1BB-costimulated CAR (4-1BB-CAR) design based on the construct used in lisocabtagene maraleucel (“liso-cel”). TM: transmembrane; ICD: intracellular domain.

FIG. 2C is a workflow diagram of CAR T-cell ex vivo generation, treatment, and culturing.

FIG. 2D and FIG. 2E show representative flow plots showing expression of type I interferon receptor (IFNAR2) on T cells before (top) or post (bottom) CAR transduction. T cells were transduced with CD28-costimulated (FIG. 2D) or 4-1BB costimulated (FIG. 2E) CARs. An isotype control antibody were used as the negative control for IFNAR2⁺ gating.

FIG. 2F and FIG. 2G show titration of IFN-α2 to CD28-costimulated CAR T cells (FIG. 2F, n=3) and 4-1BB-costimulated CAR T cells (FIG. 2G, n=3). Cells were subject to IFN-α2 for 18 hours prior to intracellular staining for IRF7, a known interferon-stimulated gene (ISG). The data points were fitted to a dose-response curve. The three IFN-I signaling conditions were labeled on the curve. Results are representative of independent experiments using CAR T cells from 2 different donors.

FIG. 2H and FIG. 2I show relative expression by quantitative reverse transcription PCR (RT-qPCR) of given ISGs in CD28-costimulated (FIG. 2H, n=3) and 4-1BB-costimulated (FIG. 2I, n=3) CAR T cells from the three culturing conditions. Expression of ISGs was normalized to expression of β-actin transcript (ACTB) and subsequently to the IFNα-neg condition. All data are shown as mean±SEM of n experimental replicates from a representative donor. Results are representative of independent experiments using CAR T cells from 2 different donors. Statistical comparisons were performed with one-way ANOVA with Tukey correction for multiple comparisons.

FIG. 2J and FIG. 2K show representative flow plots depicting gating for CAR expression (top) and T-cell lineages (bottom) in CD28-costimulated (FIG. 2J) and 4-1BB-costimulated (FIG. 2K) CAR T cells. CAR T cells were gated as live CD3⁺meGFP⁺ cells.

FIG. 2L and FIG. 2M show transduction percentage as measured by meGFP fluorescence among T cells from each culturing condition in CD28-costimulated (FIG. 2L, n=3) and 4-1BB-costimulated (FIG. 2M, n=3) CAR T cells. All data are shown as mean±SEM of n experimental replicates from a representative donor. Results are representative of independent experiments using CAR T cells from 3 different donors. Statistical analysis was performed via one-way ANOVA with Tukey correction for multiple comparisons.

FIG. 2N and FIG. 2O show stacked bar plot comparing CD8 vs CD4 proportions in CD28-costimulated (FIG. 2N, n=3) and 4-1BB-costimulated (FIG. 20, n=3) CAR T cells. All data are shown as mean±SEM of n experimental replicates from a representative donor. Results are representative of independent experiments using CAR T cells from 3 different donors. Statistical analysis was performed via two-way ANOVA with Tukey correction for multiple comparisons.

FIG. 2P and FIG. 2Q show representative flow plots depicting gating for CD45RA and CCR7 memory markers in CD28-costimulated (FIG. 2P) and 4-1BB-costimulated (FIG. 2Q) CAR T cells. Top, CD8⁺ cells, bottom, CD4⁺ T cells.

FIG. 2R and FIG. 2S show stacked bar plots comparing proportions of the SCM (CD45RA⁺CCR7⁺), CM (CD45RA⁻CCR7⁺), EM (CD45RA⁻CCR7⁻), and TE (CD45RA⁺CCR7⁻) memory phenotypes among CD8⁺ (left, n=3) and CD4⁺ (right, n=3) in CD28-costimulated (FIG. 2R) and 4-1BB-costimulated (FIG. 2S) CAR T cells. SCM, stem cell memory, CM, central memory, EM, effector memory, TE, terminally differentiated effector. All data are shown as mean±SEM of n experimental replicates from a representative donor. Results are representative of independent experiments using CAR T cells from 3 different donors. Statistical analysis was performed via two-way ANOVA with Tukey correction for multiple comparisons.

FIG. 2T and FIG. 2U show representative flow plots depicting gating for IFNAR2⁺ cells in CD28-costimulated (FIG. 2T) and 4-1BB-costimulated (FIG. 2U) CAR T cells.

FIG. 2V and FIG. 2W show expression of IFNAR2 as measured by gMFI in CD28-costimulated (FIG. 2V) and 4-1BB-costimulated (FIG. 2W) CAR T cells from the three culturing conditions. An isotype antibody was used as the negative control for IFNAR2⁺ gating. All data are shown as mean±SEM of n experimental replicates from a representative donor. Results are representative of independent experiments using CAR T cells from 2 different donors. Statistical analysis was performed via one-way ANOVA with Tukey correction for multiple comparisons.

FIG. 2X and FIG. 2Y show bar plots showing expression of co-inhibitory (PD-1, LAG-3, and TIGIT) receptors in CD28-costimulated (FIG. 2X, n=3) and 4-1BB-costimulated (FIG. 2Y, n=3) CAR T cells after ex vivo culturing. All data are shown as mean±SEM of n experimental replicates from a representative donor. Results are representative of independent experiments using CAR T cells from 3 different donors. Statistical analysis was performed via one-way ANOVA with Tukey correction for multiple comparisons.

FIG. 2Z and FIG. 2AA show direct cytotoxicity against firefly luciferase-expressing OCI-Ly8 target cells at E/T ratio of 1:1 via 6-hr bioluminescence assay (n=5). All data are shown as mean±SEM of n experimental replicates from a representative donor. Results are representative of independent experiments using CAR T cells from 3 different donors. Statistical analysis was performed via one-way ANOVA with Tukey correction for multiple comparisons.

FIG. 2AB and FIG. 2AC show intracellular staining for granzyme B (left) and perforin (right) after 4-hr of coculture with OCI-Ly8 at E/T ratio of 1:1 as measured by flow cytometry (n=3). All data are shown as mean±SEM of n experimental replicates from a representative donor. Results are representative of independent experiments using CAR T cells from 3 different donors. Statistical analysis was performed via one-way ANOVA with Tukey correction for multiple comparisons.

FIG. 2AD and FIG. 2AE show concentration of cytokines secreted by CD28-costimulated (FIG. 2AD) and 4-1BB-costimulated (FIG. 2AE) CAR T cells into the supernatant after 24-hr of coculture with OCI-Ly8 at E/T ratio of 1:1 as measured by ELISA (n=5). From left to right, IL-2, TNF, and IFNγ. All data are shown as mean±SEM of n experimental replicates from a representative donor. Results are representative of independent experiments using CAR T cells from 3 different donors. Statistical analysis was performed via one-way ANOVA with Tukey correction for multiple comparisons.

FIG. 3A and FIG. 3B show relative counts of live CAR T cells from the three culturing conditions after CAR T-cell manufacturing in CD28-costimulated (FIG. 3A, n=5) and 4-1BB-costimulated (FIG. 3B, n=6) CAR T cells. Counts were normalized to the IFNα-neg condition. All data are shown as mean±SEM of n experimental replicates from a representative donor. Results are representative of independent experiments using CAR T cells from 3 donors. Statistical analysis was performed via one-way ANOVA with Tukey correction for multiple comparisons.

FIG. 3C and FIG. 3D show proliferation of CAR T cells from the three culturing conditions in CD28-costimulated (FIG. 3C, n=3) and 4-1BB-costimulated (FIG. 3D, n=3) CAR T cells. Left, representative flow plots depicting gating for Ki-67⁺ proliferating CAR T cells at the day when IFNα was removed. Right, Ki-67⁺ proliferating cell proportions among CAR T cells and Ki-67 expression (gMFI). Non-activated T cells from human PBMCs were used as a biological control for gating. All data are shown as mean±SEM of n experimental replicates from a representative donor. Results are representative of independent experiments using CAR T cells from 3 donors. Statistical analysis was performed via one-way ANOVA with Tukey correction for multiple comparisons.

FIG. 3E and FIG. 3F show apoptosis of CAR T cells from the three culturing conditions after CAR T-cell manufacturing in CD28-costimulated (FIG. 3E, n=5) and 4-1BB-costimulated (FIG. 3F, n=6) CAR T cells. Top, representative flow plots depicting gating for viable (L/D-Annexin V⁻), early apoptotic (L/D-Annexin V⁺), and dead (L/D⁺Annexin V⁺) cells among CAR T cells. Bottom, staining for Annexin V positivity among live CAR T cells. All data are shown as mean±SEM of n experimental replicates from a representative donor. Results are representative of independent experiments using CAR T cells from 3 donors. Statistical analysis was performed via one-way ANOVA with Tukey correction for multiple comparisons.

FIG. 3G and FIG. 3H show line graphs depicting viable CAR T-cell cellularity of CD28-costimulated (FIG. 3G, n=3) and 4-1BB-costimulated (FIG. 3H, n=3) CAR T cells in vitro following coculture with OCI-Ly8 at E/T ratio of 1:5. All data are shown as mean±SEM of n experimental replicates from a representative donor. Results are representative of independent experiments using CAR T cells from 3 donors. Statistical analysis was performed via two-way ANOVA with Tukey correction for multiple comparisons.

FIG. 3I and FIG. 3J show apoptosis of CD28-costimulated CAR T cells (FIG. 3I, n=3) and 4-1BB-costimulated CAR T cells (FIG. 3J, n=3) from the three culturing conditions at day 6 after coculture with OCI-Ly8 cells at E/T ratio of 1:5. Top, representative flow plots depicting gating for viable (L/D-Annexin V⁻), early apoptotic (L/D-Annexin V⁺), and dead (L/D⁺Annexin V⁺) cells among the three culturing conditions. Bottom, staining for Annexin V positivity among live CAR T cells. All data are shown as mean±SEM of n experimental replicates from a representative donor. Results are representative of independent experiments using CAR T cells from 3 donors. Statistical analysis was performed via one-way ANOVA with Tukey correction for multiple comparisons.

FIG. 4A and FIG. 4B show cytotoxicity of CD28-costimulated (FIG. 4A, n=4) and 4-1BB-costimulated (FIG. 4B, n=4) CAR T cells. All data are shown as mean±SEM of n experimental replicates from a representative donor. Statistical analysis was performed via one-way ANOVA with Tukey correction for multiple comparisons.

FIG. 4C and FIG. 4D show concentration of cytokines secreted by CD28-costimulated (FIG. 4C, n=5) and 4-1BB-costimulated (FIG. 4D, n=5) CAR T cells into the supernatant after 24-hr of coculture with OCI-Ly8 at E/T ratio of 1:1 as measured by ELISA (n=5). From left to right, IL-2, TNF, and IFNγ. All data are shown as mean±SEM of n experimental replicates from a representative donor. Statistical analysis was performed via one-way ANOVA with Tukey correction for multiple comparisons.

FIG. 4E and FIG. 4F show line graphs depicting viable CAR T-cell cellularity of CD28-costimulated (FIG. 4E, n=3) and 4-1BB-costimulated (FIG. 4F, n=3) CAR T cells in vitro following coculture with OCI-Ly8 at E/T ratio of 1:5. All data are shown as mean±SEM of n experimental replicates from a representative donor. Statistical analysis was performed via two-way ANOVA with Tukey correction for multiple comparisons.

FIG. 4G and FIG. 4H show apoptosis of CD28-costimulated CAR T cells (FIG. 4G, n=3) and 4-1BB-costimulated CAR T cells (FIG. 411, n=3) from the three culturing conditions at the time points when cellularity declined in (FIG. 4E) and (FIG. 4F): day 9 for (FIG. 4G) and day 6 for (FIG. 411) at an E:T ratio of 1:5. All data are shown as mean±SEM of n experimental replicates from a representative donor. Statistical analysis was performed via one-way ANOVA with Tukey correction for multiple comparisons.

FIG. 5A is a schematic depicting timeline for NSG mouse model based on OCI-Ly8 lymphoma cell line. Seven days following OCI-Ly8 injection, mice were randomly divided into groups that were treated with either PBS or CAR T cells from the three culturing conditions.

FIG. 5B, FIG. 5C, and FIG. 5D show Kaplan-Meier analysis of survival of OCI-Ly8-bearing mice treated with PBS or 5×10⁵ CD28-costimulated CAR T cells from various culture conditions. CAR T cells were generated from donor 1 (FIG. 5B, n=8, 12, 13, 8), donor 2 (FIG. 5C, n=5, 5, 4, 4), and donor 3 (FIG. 5D, n=4, 6, 6, 6). Statistical analysis was performed by log-rank (Mantel-Cox) test.

FIG. 5E and FIG. 5F show Kaplan-Meier analysis of survival of OCI-Ly8-bearing mice treated with PBS or 5×10⁵ (FIG. 5E) or 1×10⁶ (FIG. 5F) 4-1BB-costimulated CAR T cells from various culture conditions. CAR T cells were generated from donor 4 (FIG. 5E, n=6, 6, 6, 6), and donor 5 (FIG. 5F, n=4, 5, 5, 5). Statistical analysis was performed by log-rank (Mantel-Cox) test.

FIG. 5G is representative bioluminescence imaging of tumor burden in mice treated with PBS or CD28-costimulated CAR T cells generated from donor 1 in FIG. 5B under the three IFNα culturing conditions, and the corresponding quantification of bioluminescence signals (n=5, 5, 5, 4).

FIG. 5H is representative bioluminescence imaging of tumor burden in mice treated with PBS or CD28-costimulated CAR T cells generated from donor 3 in FIG. 5D under the three IFNα culturing conditions, and the corresponding quantification of bioluminescence signals (n=4, 6, 6, 6).

FIG. 5I is representative bioluminescence imaging of tumor burden in mice treated with PBS or 4-1BB-costimulated CAR T cells generated from donor 4 in FIG. 5E under the three IFNα culturing conditions, and the corresponding quantification of bioluminescence signals (n=6, 6, 6, 6).

FIG. 6A and FIG. 6B show direct cytotoxicity of CD28-costimulated (FIG. 6A, n=3) and 4-1BB-costimulated (FIG. 6B, n=3) CAR T cells against firefly luciferase-expressing NALM6 target cells at E/T ratio of 1:1 via 6-hr bioluminescence assay. Statistical analysis was performed via one-way ANOVA with Tukey correction for multiple comparisons. All data are shown as mean±SEM of n experimental replicates from a representative donor. Results are representative of independent experiments using CAR T cells from 3 different donors.

FIG. 6C and FIG. 6D show direct cytotoxicity of CD28-costimulated (FIG. 6C, n=3) and 4-1BB-costimulated (FIG. 6D, n=3) CAR T cells against firefly luciferase-expressing VAL target cells at E/T ratio of 1:1 via 6-hr bioluminescence assay. Statistical analysis was performed via one-way ANOVA with Tukey correction for multiple comparisons. All data are shown as mean±SEM of n experimental replicates from a representative donor. Results are representative of independent experiments using CAR T cells from 2 different donors.

FIG. 6E and FIG. 6F show line graphs depicting viable CD28-costimulated (FIG. 6E, n=3) and 4-1BB-costimulated (FIG. 6F, n=3) CAR T-cell cellularity in vitro following coculture with NALM6 at E/T ratio of 1:5. Statistical analysis was performed via two-way ANOVA with Tukey correction for multiple comparisons. All data are shown as mean±SEM of n experimental replicates from a representative donor. Results are representative of independent experiments using CAR T cells from 3 different donors.

FIG. 6G and FIG. 6H show line graphs depicting viable CD28-costimulated (FIG. 6G, n=3) and 4-1BB-costimulated (FIG. 6H, n=3) CAR T-cell cellularity in vitro following coculture with VAL at E/T ratio of 1:5. Statistical analysis was performed via two-way ANOVA with Tukey correction for multiple comparisons. All data are shown as mean±SEM of n experimental replicates from a representative donor. Results are representative of independent experiments using CAR T cells from 2 different donors.

FIG. 6I and FIG. 6J show apoptosis of CD28-costimulated (FIG. 6I, n=3) and 4-1BB-costimulated (FIG. 6J, n=3) CAR T cells from the three culturing conditions at the time points when cellularity declined in (FIG. 6E) and (FIG. 6F). Day 6 and day 3 after coculture, respectively. Statistical analysis was performed via one-way ANOVA with Tukey correction for multiple comparisons. All data are shown as mean±SEM of n experimental replicates from a representative donor. Results are representative of independent experiments using CAR T cells from 3 different donors.

FIG. 6K and FIG. 6L show apoptosis of CD28-costimulated (FIG. 6K, n=3) and 4-1BB-costimulated (FIG. 6L, n=3) CAR T cells from the three culturing conditions at the time points when cellularity declined in (FIG. 6G) and (FIG. 6H). Day 9 and day 3 after coculture, respectively. Statistical analysis was performed via one-way ANOVA with Tukey correction for multiple comparisons. All data are shown as mean±SEM of n experimental replicates from a representative donor. Results are representative of independent experiments using CAR T cells from 2 different donors.

FIG. 6M is a schematic depicting a timeline for testing a NSG mouse model based on NALM6 leukemia cell line.

FIG. 6N and FIG. 6O show Kaplan-Meier analysis of survival of NALM6-bearing mice treated with PBS, 5×10⁵ CD28-costimulated CAR T cells (FIG. 6N, n=8, 8, 8, 8) or 1×10⁶ 4-1BB-costimulated CAR T cells (FIG. 6O, n=8, 9, 9, 9), generated from a healthy donor under the three IFNα culturing conditions. The PBS-treated mice in FIG. 6N and FIG. 6O were the same cohort and were used as a shared control in the same experiment. Statistical analysis was performed by log-rank (Mantel-Cox) test.

FIG. 6P and FIG. 6Q show representative bioluminescence imaging of tumor burden in mice shown in FIG. 6N and FIG. 6O respectively, with corresponding quantification of bioluminescence.

FIG. 7A is a schematic depicting timeline for an OCI-Ly8 tumor second challenge model.

FIG. 7B shows a Kaplan-Meier analysis of survival of OCI-Ly8-bearing mice treated with PBS or 3×10⁶ CD28-costimulated CAR T cells generated from a healthy donor under 2 IFNα culturing conditions (n=4, 7, 7). Statistical analysis was performed by log-rank (Mantel-Cox) test.

FIG. 7C shows representative bioluminescence imaging of tumor burden in mice shown in FIG. 7B, along with additional CAR-treated mice used for spleen collection (n=4, 12, 12).

FIG. 7D shows corresponding quantification of bioluminescence signals for mice in FIG. 7C without mice used for spleen collection (n=4, 7, 7).

FIG. 7E is a longitudinal quantification of CAR⁺ cells in 50 μL peripheral blood from mice in FIG. 7C. Statistical analysis was performed by two-way ANOVA with Tukey correction for multiple comparisons.

FIG. 7F shows quantification of CAR⁺ cells in the spleen collected at day 27 from mice in FIG. 7C. Statistical analysis was performed by unpaired t-test.

FIG. 7G and FIG. 7H show proportion of the SCM (CD45RA⁺CD62L⁺), CM (CD45RA⁻CD62L⁺), EM (CD45RA⁻CD62L⁻), and TE (CD45RA⁺CD62L⁻) memory phenotypes among CD8⁺ and CD4⁺ CAR T cells in spleen of mice shown in FIG. 7F. Statistical analysis was performed by two-way ANOVA with Tukey correction for multiple comparisons.

FIG. 7I is a cartoon summarizing effects of IFN-I signaling on CAR T cells at different doses. IFN-lo CAR T cells exhibit reduced cytotoxicity compared to IFN-hi CAR T cells, but they also exhibit significantly greater persistence and in vivo efficacy making them a more effective and useful treatment tool.

### DETAILED DESCRIPTION

It is to be understood that the particular aspects of the specification described herein are not limited to the specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

All publications, patents and patent applications cited herein are hereby expressly incorporated by reference in their entirety for all purposes.

### Definitions

Before describing the methods and compositions of the disclosure in detail, a number of terms will be defined. As used herein, the singular forms “a,” “an,” and “the” include plural referents unless the context clearly dictates otherwise. For example, reference to “a therapeutic target” means one or more therapeutic targets.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the methods and compositions as described herein or to imply that certain features are critical, essential, or even important to the structure or function of the subject matter recited in the claims.

As used herein, the term “level of expression” or “expression level” refers to a measurable level of expression of the products of biomarkers, such as, without limitation, the level of messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

As used herein, the term “differentially expressed” or “differential expression” refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example, as measured by the amount of messenger RNA transcript and/or the amount of protein in a sample as compared with the measurable expression level of a given biomarker in a control.

As used herein, the term “control” or “control sample” refers to a sample or a specific group of samples or experimental results that is used as a standard or a baseline to compare against a sample or a group of samples or experimental results to which a treatment or other set of factors being investigated is applied. The control or control sample has not been treated with the treatment or other factors being investigated. In this way, potential effects of a treatment or other factors being investigated can be objectively determined by observing differences between a treated sample and a control or control sample (e.g., an “untreated sample” or “untreated control”). For example, in various aspects described herein, a control can be determined from biological sample(s) from healthy individuals/populations.

As used herein, “sample” or “biological sample” is referred to in its broadest sense, and includes solid and liquid or any biological sample obtained from nature, including an individual, body fluid, cell line, tissue culture, or any other source, which may contain genetic material.

Unless otherwise defined, all technical terms used herein have the same meaning as commonly understood by a person of ordinary skill in the art to which this disclosure belongs.

### Overview

The present disclosure relates to populations of interferon (IFN)-enhanced chimeric antigen receptor (CAR)-T cells and methods of their production. The methods comprise treating a population of CAR-T cells expressing a CAR gene construct with a concentration of an IFN receptor agonist sufficient to produce a population of IFN-enhanced CAR-T cells, wherein compared to an untreated control CAR-T cell population, the population of IFN-enhanced CAR-T cells exhibits increased expression of one or more interferon-stimulated genes, as well as increased persistence and in vivo efficacy. The disclosure also relates to a pharmaceutical composition comprising the population of IFN-enhanced chimeric antigen receptor (CAR)-T cells and methods of treating cancer with the pharmaceutical composition.

### Methods of Producing IFN-Enhanced CAR-T Cells

The present disclosure provides a method of preparing a CAR-T cell population, comprising:
treating a population of CAR-T cells expressing a CAR gene construct with an interferon (IFN) receptor agonist to produce a population of IFN-enhanced CAR-T cells,
wherein compared to an untreated control CAR-T cell population, the population of IFN-enhanced CAR-T cells exhibits increased expression of one or more interferon-stimulated genes.

Also provided herein is a method of producing a population of interferon (IFN)-enhanced chimeric antigen receptor (CAR)-T cells, comprising:
a) introducing a CAR gene construct into a population of T cells to produce a population of CAR-T cells; and
b) co-culturing the population of CAR-T cells with a concentration of an IFN receptor agonist to obtain a population of IFN-enhanced CAR-T cells that exhibit about 40% to about 90% maximal expression of an interferon-stimulated gene (ISG).

As shown in Example 1, by investigating the cell infusion products (IPs) of CAR T-cell therapy patients with r/r DLBCL using single-cell transcriptomics, compared to patients with progressive disease, the IPs of complete responders demonstrated stronger signatures of type I interferon (IFN-I) signaling and cytotoxicity. As potent immunomodulatory cytokines, IFN-I have long been of interest for holding significant potential to modulate immunotherapy response rates (Crouse et al., 2015, Nature Reviews Immunology 15, 231-242; Musella et al., 2017, Oncoimmunology 6, 1-12). Upon binding to type I interferon receptors, IFN-I cytokines trigger a phosphorylation cascade that activates transcription factors such as signal transducer and activator of transcription 1 (STAT1), STAT2, and interferon regulatory factor 9 (IRF9) (Crouse et al., 2015, Nature Reviews Immunology 15, 231-242). These transcription factors coordinate transcription of interferon-stimulated genes, such as OAS1/2/3, OASL, ADAR, IFITM1/2/3, IFI6, IFI16, ZAP, APOBEC3, ISG20, SOCS1, IRF7, MX1, ADAR, and ISG15, which mediate antiviral immunity and effector functions (Crouse et al., 2015, Nature Reviews Immunology 15, 231-242; Ning et al., 2011, Genes and Immunity 12, 399-414). In CD8+ T cells, IFN-I signaling enhances cytotoxicity (Curtsinger et al., 2005, J. Immunol. 174, 4465-4469) and induces central memory differentiation (Ramos et al., 2009, Blood 113, 5516-5525), while also controlling T-cell exhaustion (Lukhele et al., 2022, Immunity 55, 2369-2385.e10), inhibitory receptor expression (Sumida et al., 2022, Nat. Immunol. 23, 632-642), and apoptosis (Harrer et al., 2022, Cells 11, 3839; Evgin et al., 2020, Nat. Commun. 11, 1-15). Due to their immunomodulatory effects, clinical formulations of IFN-I cytokines (e.g., Avonex™, Intron™ A, Alferon™ N) have been approved by the FDA for the treatment of malignancies (e.g., hairy cell leukemia, follicular lymphoma) (Musella et al., 2017, Oncoimmunology 6, 1-12), autoimmune diseases (e.g., multiple sclerosis) (Jakimovski et al., 2018, Cold Spring Harb. Perspect. Med. 8, 1-19), and viral infections (e.g., hepatitis B and C) (Li et al., Cellular Physiology and Biochemistry 51, 2377-2396). However, despite being an attractive FDA-approved cytokine that can regulate T-cell immune functions in cancer, autoimmunity, and infection, IFN-I has not yet been utilized to modulate CAR T-cell therapy response rates.

The present disclosure establishes IFN receptor agonists such as IFN cytokines as enhancers during the CAR T-cell manufacturing process to address CAR T-cell therapy failure rates. Through in vitro functional assays and in vivo DLBCL xenograft mouse models, a novel ex vivo manufacturing method has been discovered that leverages low-strength IFN cytokines to improve the in vivo treatment efficacy of CAR T-cell therapy.

As used herein, the term “IFN-enhanced CAR-T cells” refers to CAR-T cells with effector functions superior to CAR-T cells produced without co-culturing with an IFN receptor agonist. As shown in Example 2, compared to CAR-T control cells without IFN enhancement, IFN-enhanced CAR-T cells that have been cultured with “IFN-α lo” (which is a concentration of IFN receptor agonist IFN-α that elicits 40% to 65% maximal expression of IRF7) and with “IFN-α hi” (which is a concentration of IFN receptor agonist IFN-α that elicits over 65% maximal expression of IRF7), are more effective in killing tumor cells by increased the CAR-T cell cytotoxicity, increased expression of cytolytic molecules, and increased secretion of cytokines. However, unexpected therapeutic advantages were realized by using IFN-α lo versus IFN-α hi concentrations when manufacturing IFN-enhanced CAR-T cells, as described below.

As used herein, the term “introducing a gene into” a cell or a population of cells refers to incorporating the gene into cells by methods that are known in the art such as transduction or transfection by chemical, physical, or biological method. The CAR gene construct can be encapsulated in a viral or non-viral vector to be delivered to the cells. Examples of a viral vector include, but are not limited to, a lentivirus or a retrovirus. In some embodiments, the viral vector is a lentivirus. Examples of a non-viral vector includes, but is not limited to, a transposon, an mRNA molecule, a liposome, a molecular conjugate, or a lipid nanoparticle. The CAR gene construct encodes a CAR that binds to a therapeutic cell surface antigen. The target specificity and the design of a CAR construct can vary depending on the target cells and the disease. The Examples show that the disclosed method utilizing an IFN agonist can enhance CAR T Cells which are generated using two different constructs of anti-CD19 CAR (axi-cel and liso-cel), demonstrating that IFN-enhanced CAR T cells can be produced according to the disclosed method regardless of the CAR design. In some embodiments, the cell surface antigen includes, but is not limited to, CD19, CD20, CD22, or BCMA. In some embodiments, the CAR gene construct is an axicabtagene ciloleucel construct or a lisocabtagene maraleucel construct.

As used herein, the term “agonist” refers a molecule that activates biological signaling from a receptor. In some embodiments, the interferon (IFN) receptor can be an interferon receptor for type I interferon (IFN-I) or an interferon receptor for type III interferon (IFN-III). IFN receptors are found in immune cells and elsewhere, and made up of multiple transmembrane polypeptides and protein tyrosine kinases (Lazear et al., 2019, Immunity 50, 907-923). IFN-I receptor is a heterodimer of IFNAR1-encoded a/P receptor subunit 1 and IFNAR2-encoded a/P receptor subunit 2. IFN-III receptor is a heterodimer of IFN-LR1-encoded X receptor subunit 1 and IL-10R2-encoded interleukin 10 receptor subunit 2. IFN-I receptors can be activated by type I interferon cytokines such as IFN-α, IFN-β, IFN-ε, IFN-κ, IFN-ω, or a combination thereof. IFN-III receptors can be activated by type III interferon cytokines such as IFN-λ1, IFN-λ2, IFN-λ3, IFN-λ4, or a combination thereof. In some embodiments, the IFN-1 receptor agonist is a synthetic IFN-α2. In some embodiments, the concentration of the IFN-α2 is between about 100 IU/mL and about 1000 IU/mL. In some embodiments, the concentration of the IFN-α2 is about 100 IU/mL, 200 IU/mL, 300 IU/mL, 400 IU/mL, 500 IU/mL, 600 IU/mL, 700 IU/mL, 800 IU/mL, 900 IU/mL, or 1000 IU/mL. In some embodiments, the concentration of the IFN-α2 is about 300 IU/mL.

It should be noted that IFN receptor agonists have varied strength of binding (affinity and efficacy) to the receptors. Therefore, the concentration of the agonist needs to be titrated to determine the appropriate concentration that can elicit 40% to 70% of the maximal activation of the receptor. In some embodiments, activation of the receptor can be measured by the expression of the interferon-stimulated gene (ISG). As used herein, the term “interferon-stimulated gene” refers to a gene that is expressed at a level (for example, upregulated) that depends on interferon signaling pathway that depends on the activation of IFN receptor.

In some embodiments, the population of T cells is isolated from peripheral blood mononuclear cells (PBMCs) obtained from donors. In some embodiments, about 300 IU/mL IFN-α2 can elicit about 40% maximal expression of ISG in T cells from one donor and about 70% maximal expression of ISG in T cells from another donor. The heterogeneity between donors needs to be accounted for determining an appropriate concentration of IFN receptor agonist to be used in the disclosed methods.

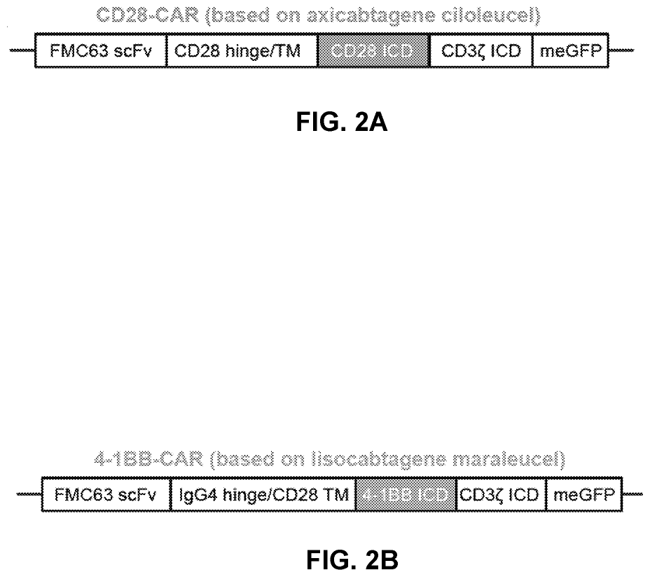

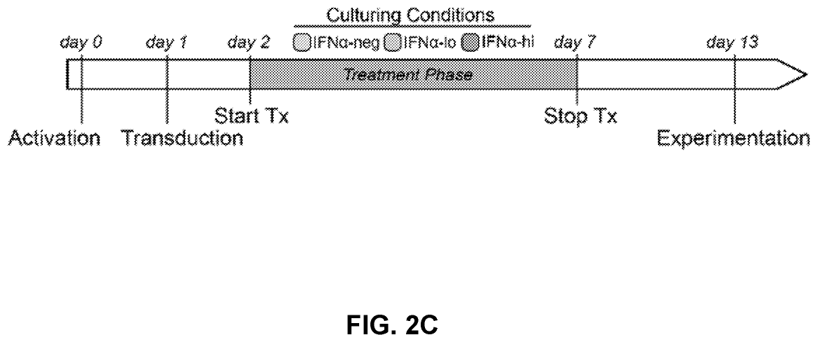

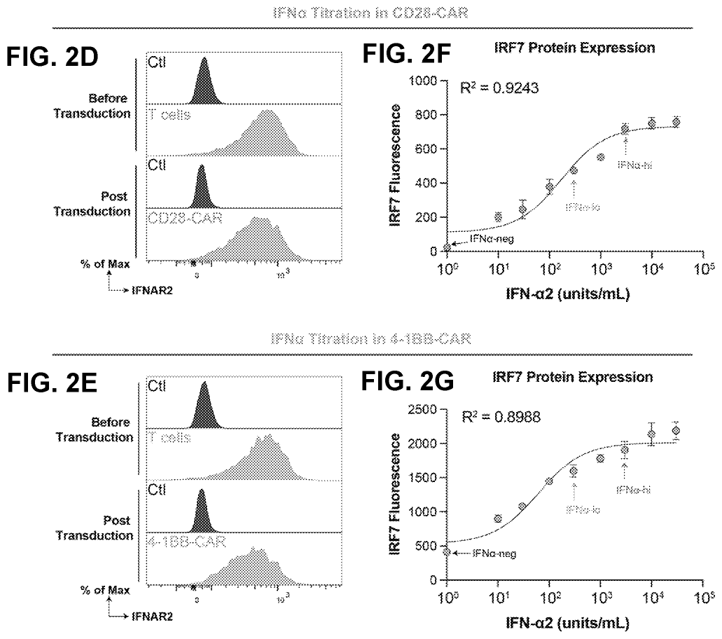

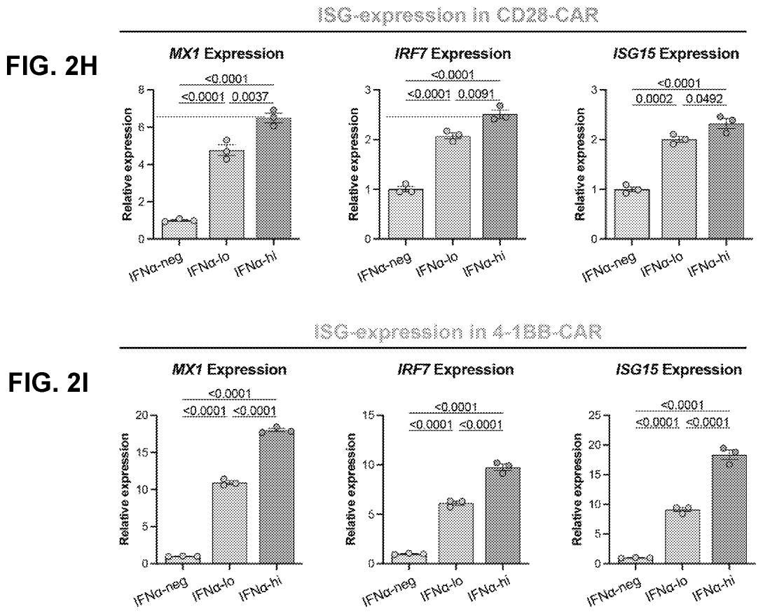

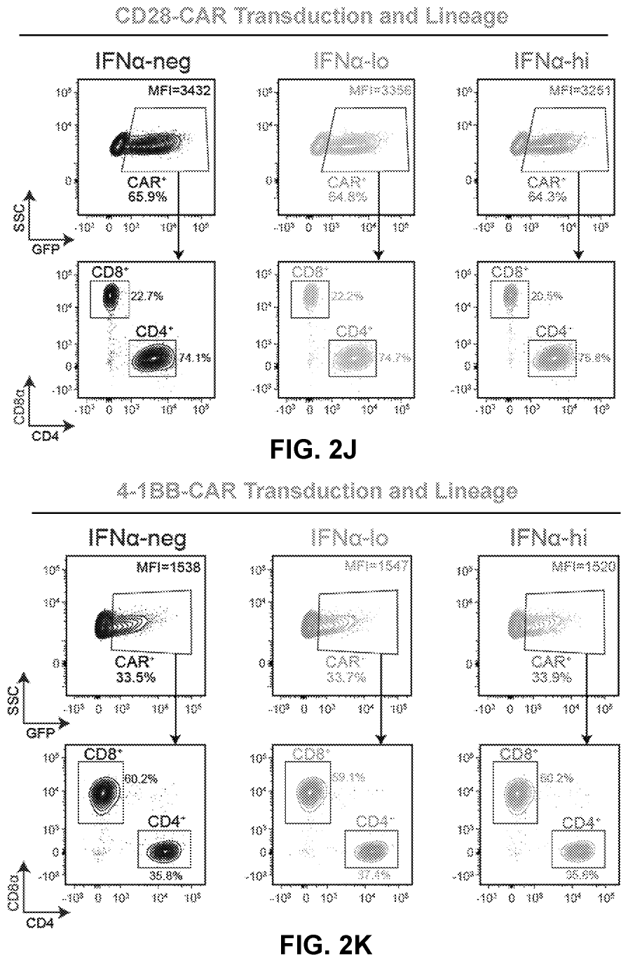

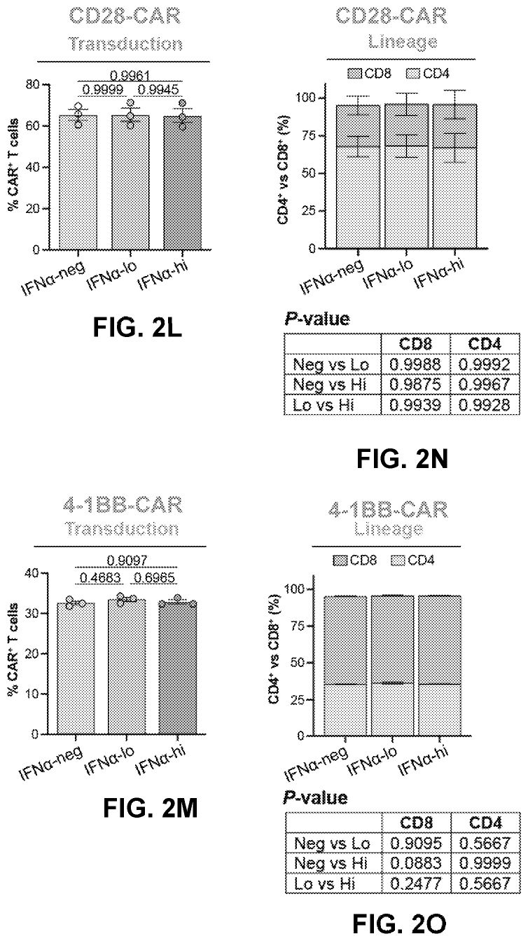

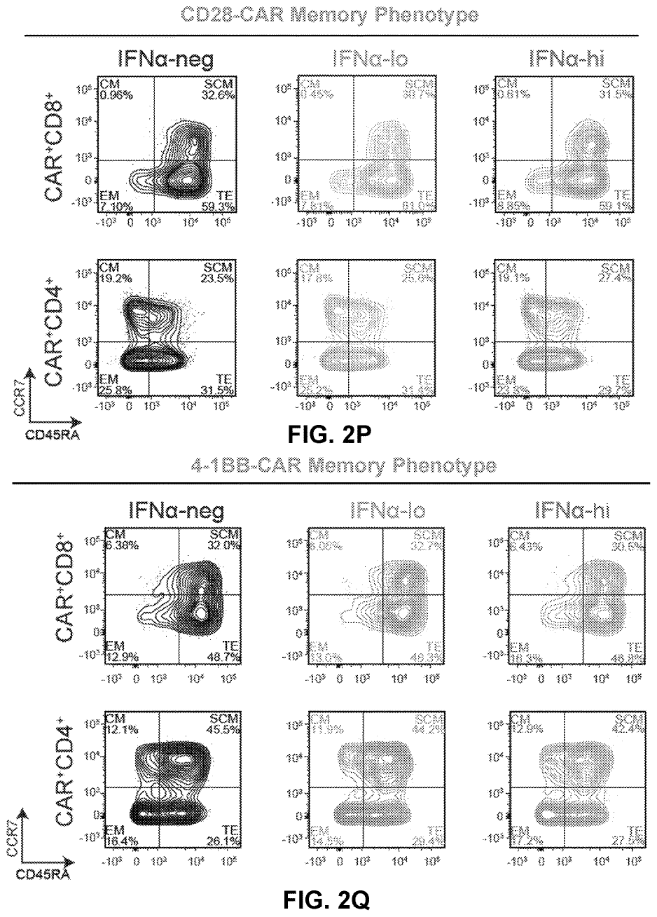

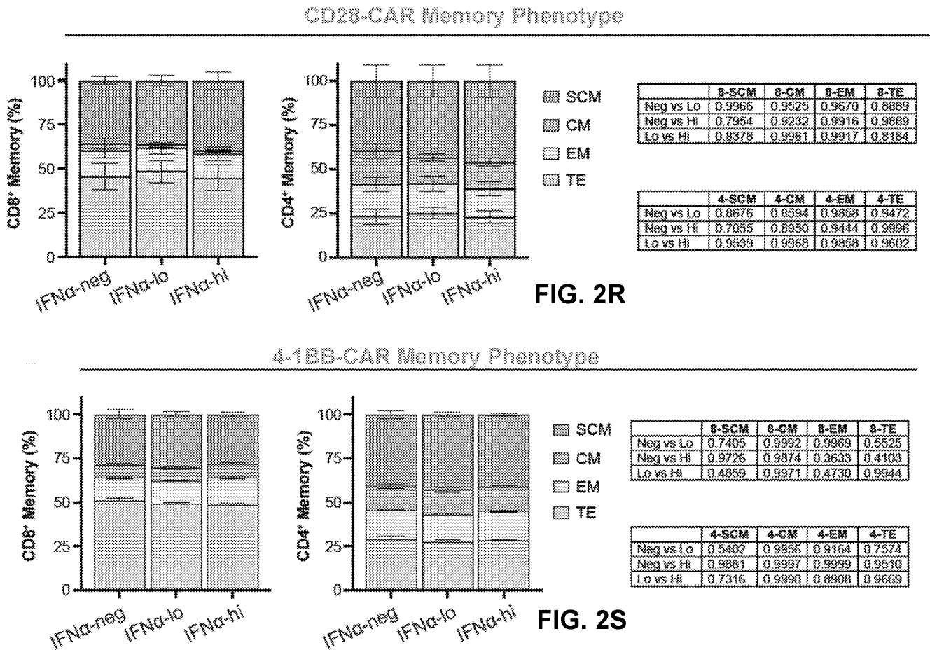

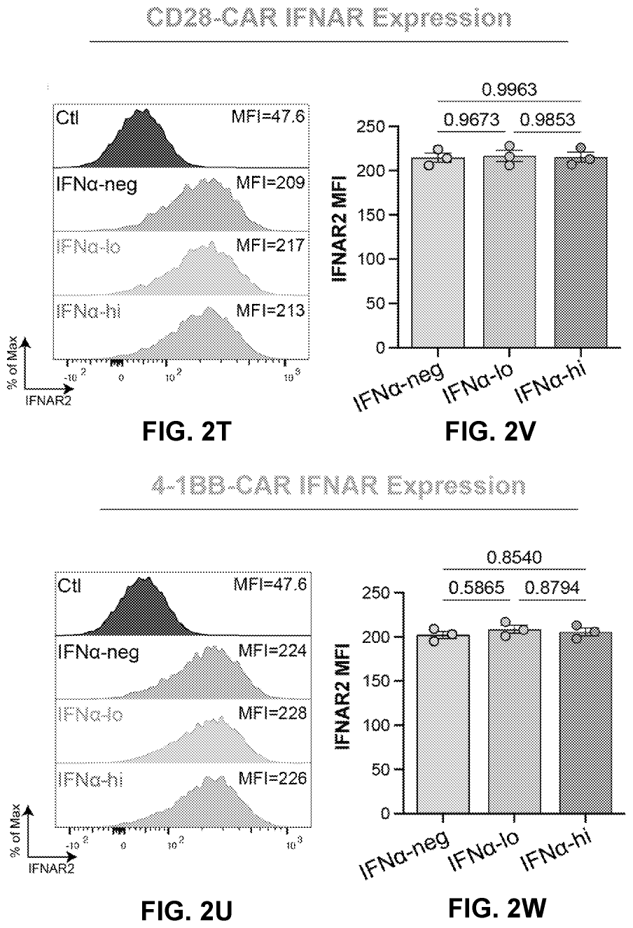

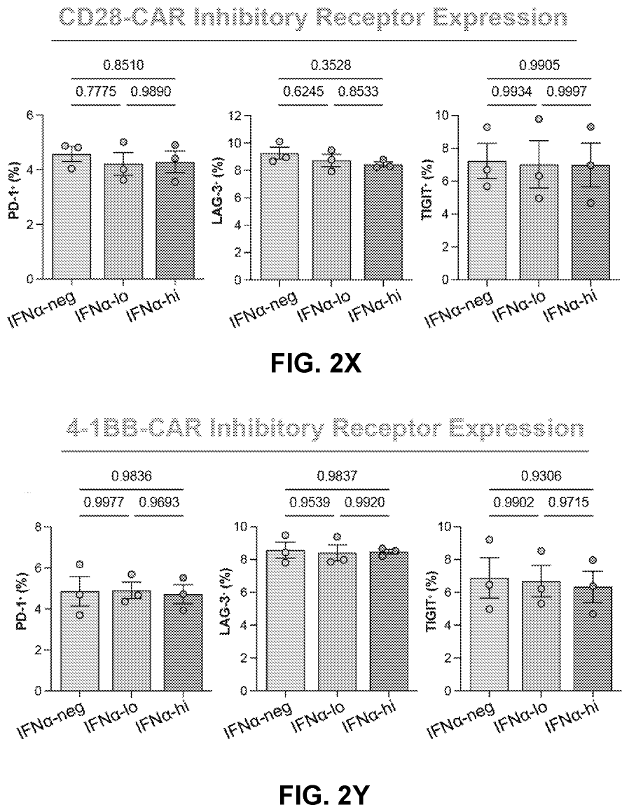

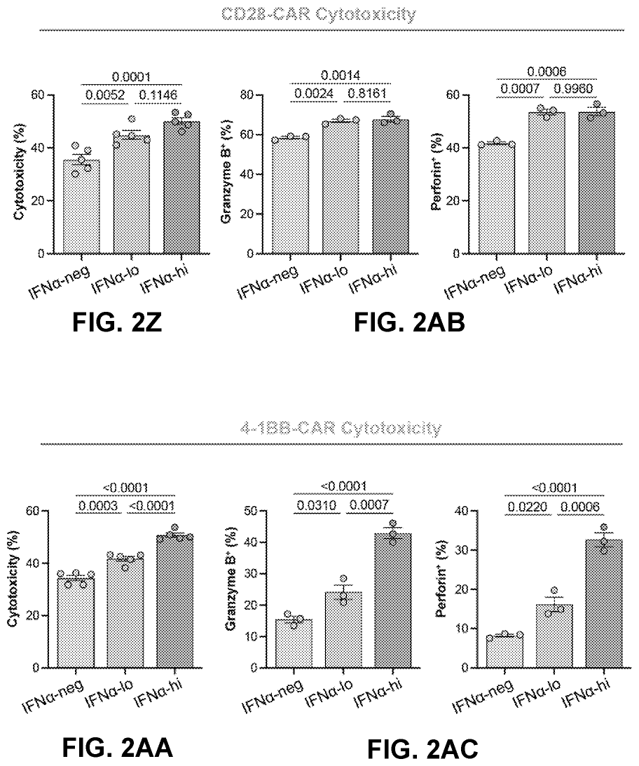

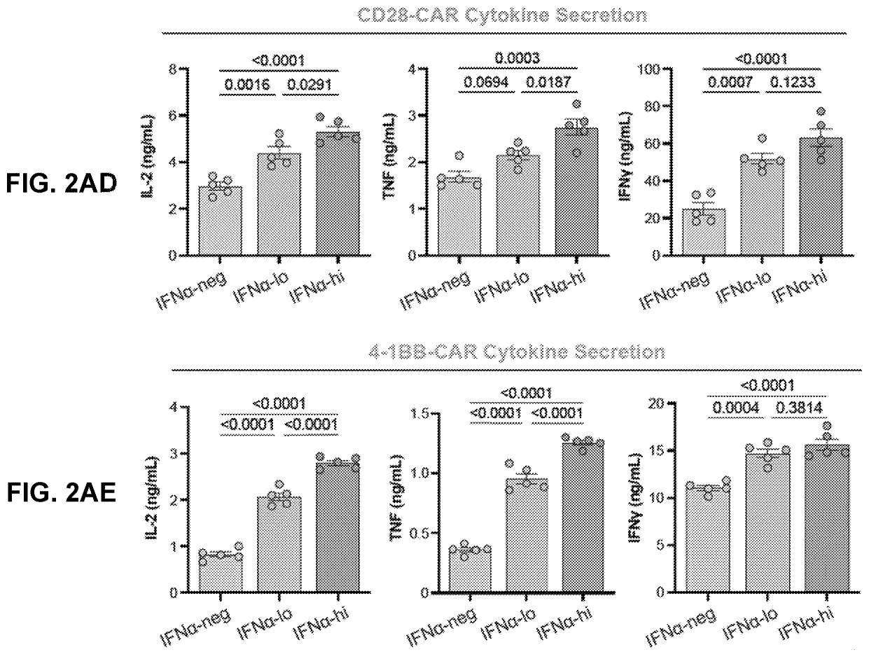

As shown in Examples 2-7, the concentration of the IFN receptor agonist is important for enhancing IFN signaling in CAR-T cells without increasing apoptosis of the CAR-T cells. As used herein, the term “low-strength IFN signaling” refers to 40% to 70% of maximal activation of IFN signaling pathway as measured by the IFN signaling's effector function. Similarly, “high-strength IFN signaling” refers to over 70% of maximal activation of IFN signaling pathway as measured by the IFN signaling's effector function. In this disclosure, the IFN signaling's effector function is measured by the expression of ISG. Examples of ISGs include, but not limited to, OAS1/2/3, OASL, ADAR, IFITM1/2/3, IFI6, IFI16, ZAP, APOBEC3, ISG20, SOCS1, IRF7, MX1, and ISG15. In FIG. 2F and FIG. 2G for examples, about 300 IU/mL IFN-α2, a concentration that provoke low-strength IFN signaling, can elicit about 66.7% maximal expression of IRF7. As used herein, the term “maximal expression” refers to the highest expression of ISG that can be induced by an IFN receptor agonist. Maximal expression can be deduced by a dose response curve (ISG expression vs IFN receptor agonist concentration) as shown in FIG. 2F and FIG. 2G.

In some embodiments, the disclosed method further comprises activating T cell receptor (TCR) signaling in the population of T cells or in the population of CAR-T cells before co-culturing the population of T cells or CAR-T cells with an IFN receptor agonist in step (b). As used herein, the term “TCR receptor signaling” refers to activation of TCR to promote a number of signaling cascades that ultimately determine cell fate through regulating cytokine production, cell survival, proliferation, and differentiation (Mariuzza et al., 2020, J Biol Chem. 295, 914-925). Activation of T lymphocytes is a key event for an efficient response of the immune system. TCR signaling can be activated by anti-CD3 antibody, anti-CD28 antibody, peptide-loaded major histocompatibility complex (pMHC), phorbol myristate acetate (PMA) with ionomycin, peptide loaded dendritic cells, or other molecules that are known in the arts. In some embodiments, the TCR signaling is activated by anti-CD3 antibody and/or anti-CD28 antibody.

### IFN-Enhanced CAR-T Cells

The present disclosure provides a population of IFN-enhanced CAR-T cells that is produced from the disclosed methods. Also provided herein is a population of IFN-enhanced CAR-T cells having between 40% and 70% of maximal expression of one or more interferon-stimulated genes induced by treatment with an IFN receptor agonist.

In some embodiments, the ISG is OAS1/2/3, OASL, ADAR, IFITM1/2/3, IFI6, IFI16, ZAP, APOBEC3, ISG20, SOCS1, IRF7, MX1, or ISG15, or a combination thereof. The population of IFN-enhanced CAR-T cells is distinct from T cells found in nature due to expression of synthetic CAR. Additionally, the population of IFN-enhanced CAR-T cells have higher expression of one or more ISGs compared to a control population of CAR-T cells not treated with an IFN receptor agonist. In some embodiments, the IFN-enhanced CAR-T cells express an axicabtagene ciloleucel CAR gene construct or a lisocabtagene maraleucel CAR gene construct. In these embodiments, the IFN receptor agonist is IFN-α2 and the IFN-α2 concentration is between about 100 IU/mL and about 1000 IU/mL. In certain embodiments, the IFN-α2 concentration is about 300 IU/mL and the interferon-stimulated gene is IRF7, wherein the population of IFN-enhanced CAR-T cells having between 40% and 70% of maximal IRF7 expression.

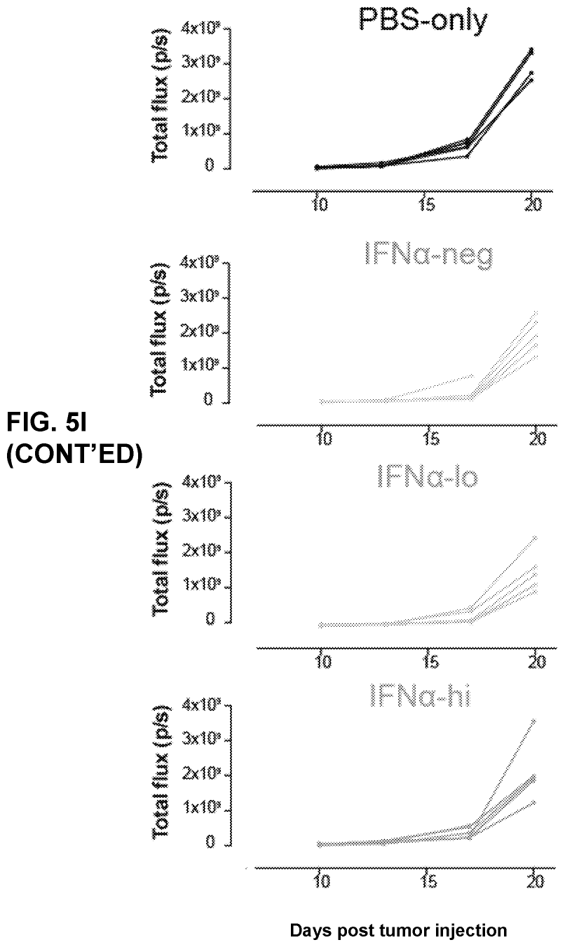

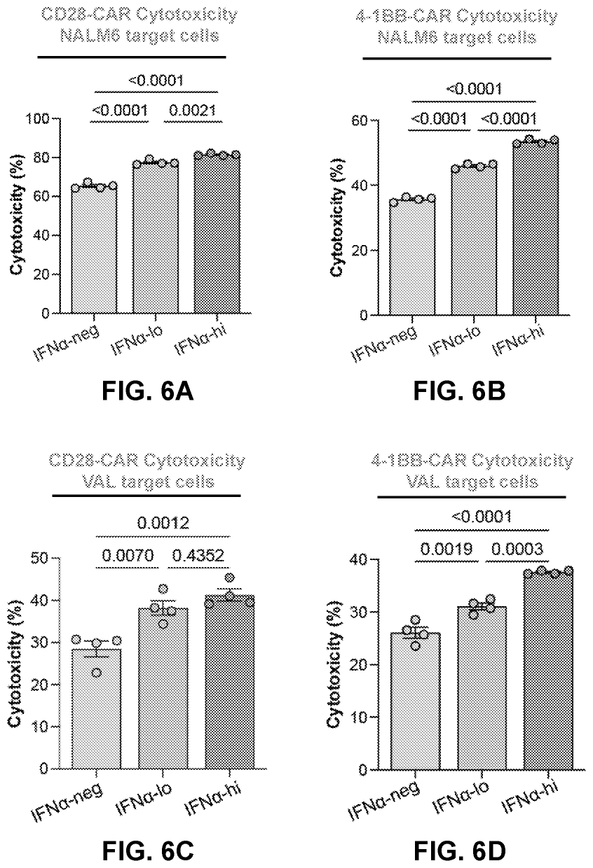

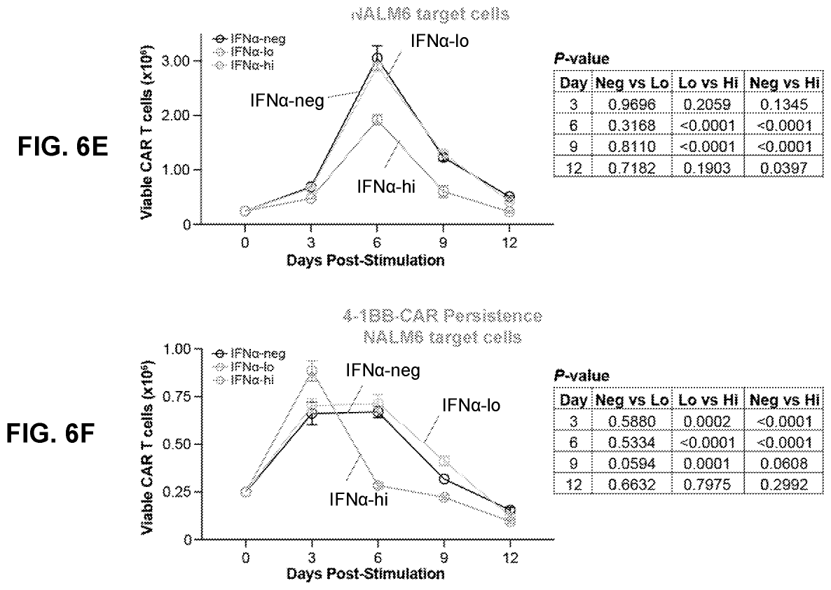

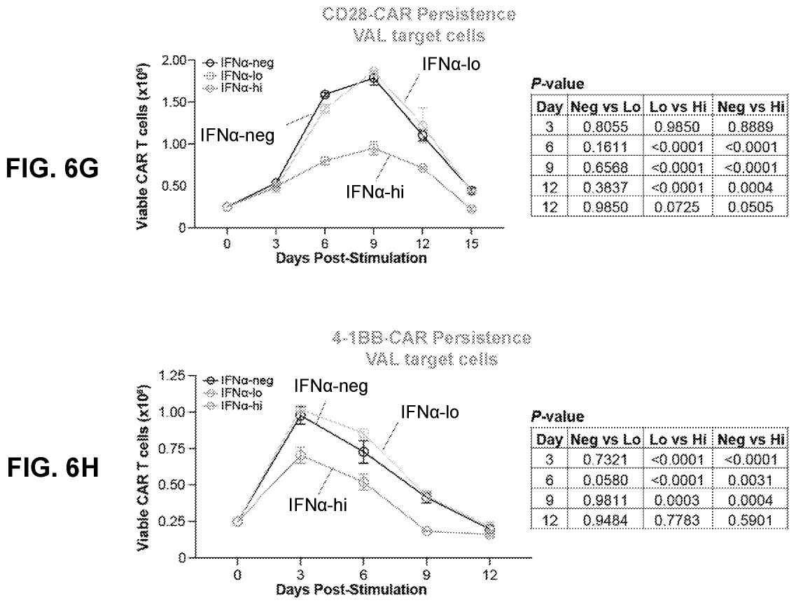

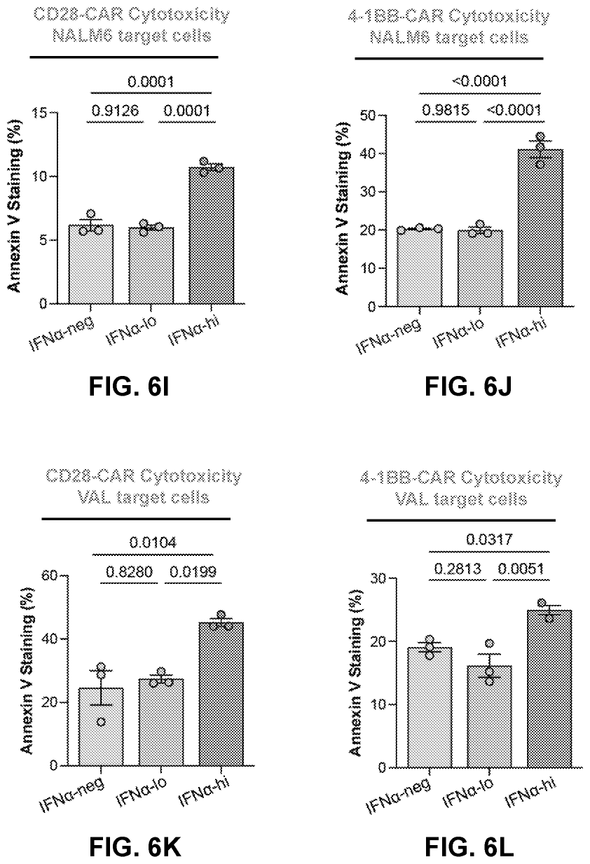

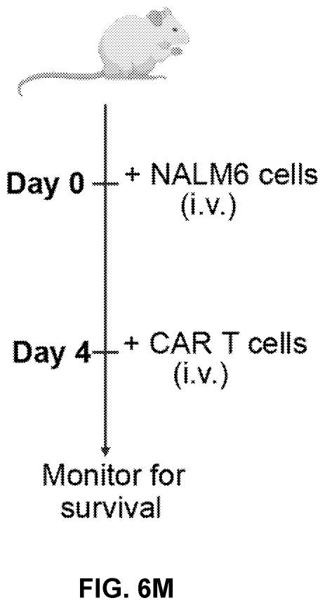

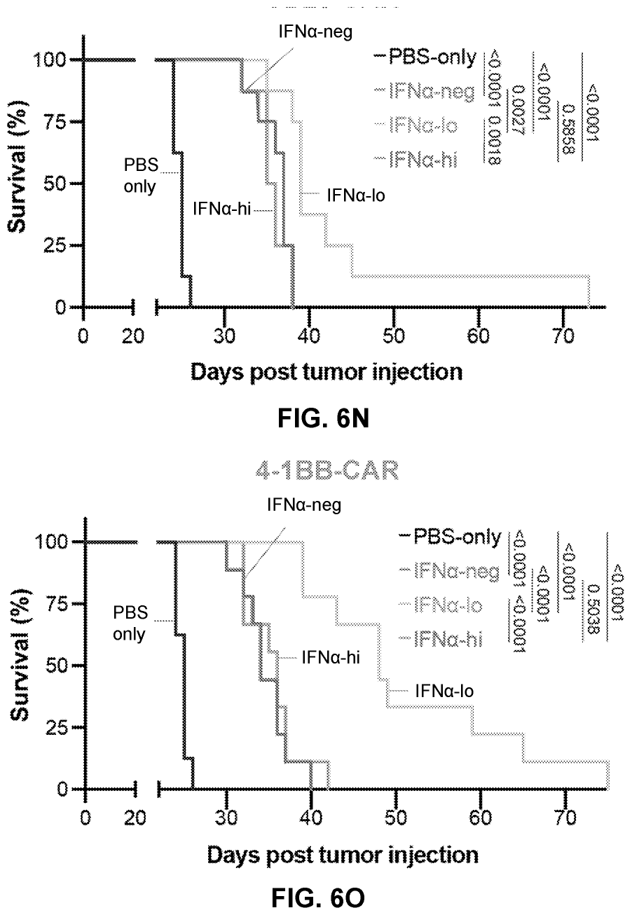

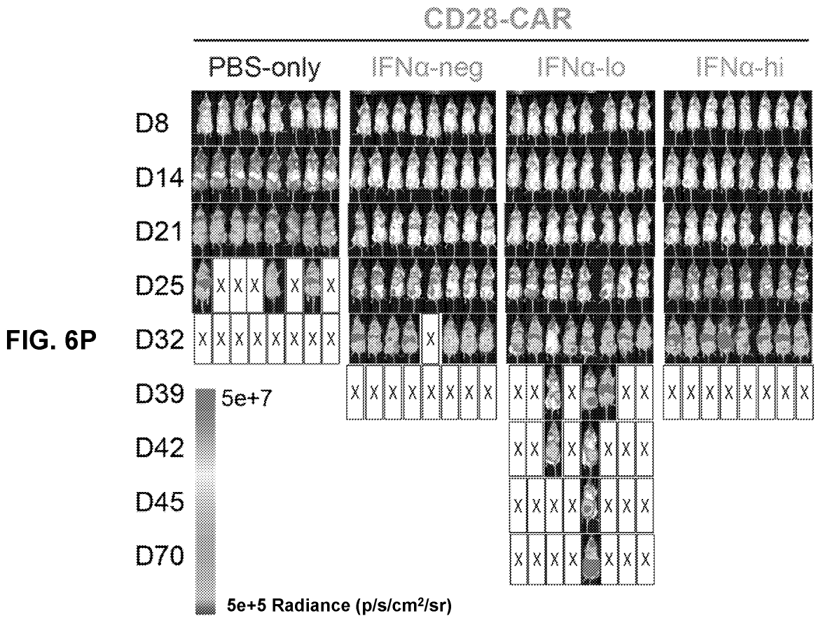

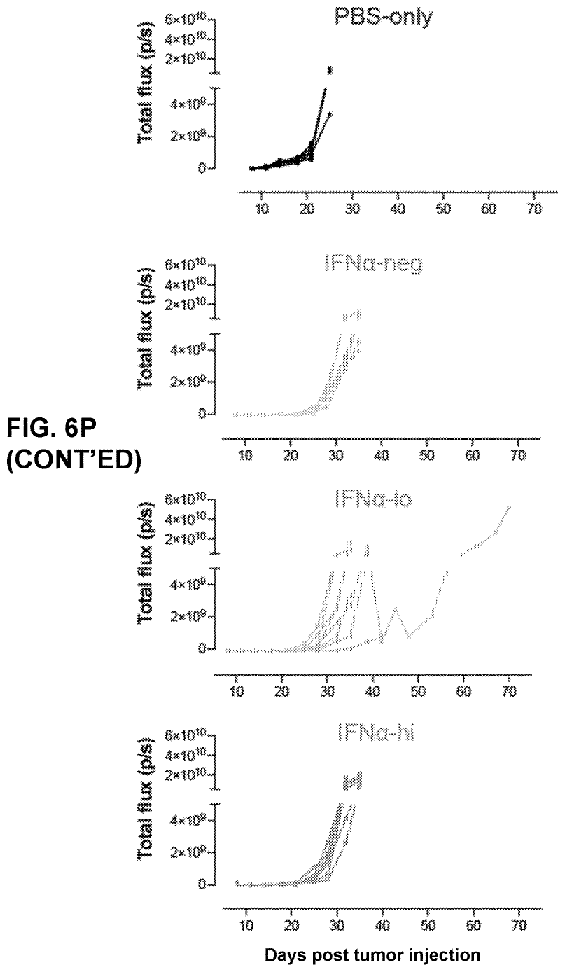

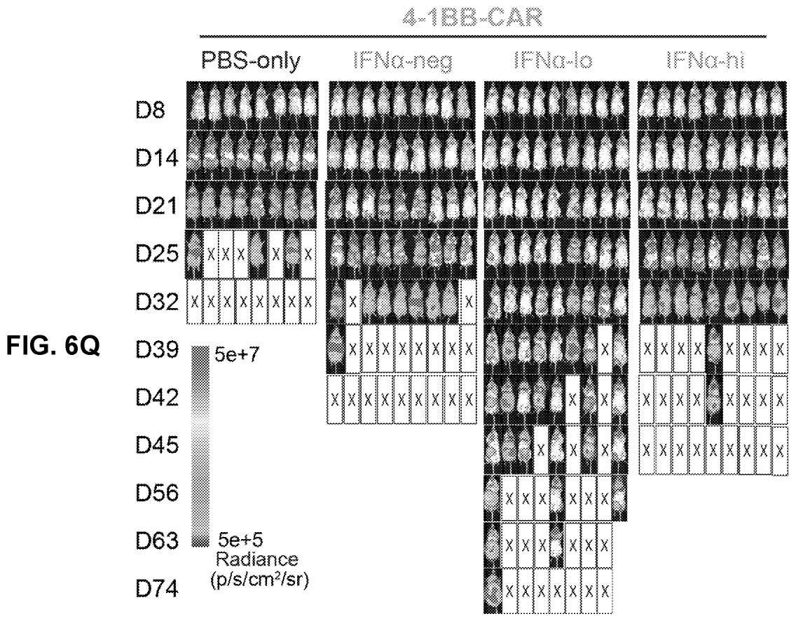

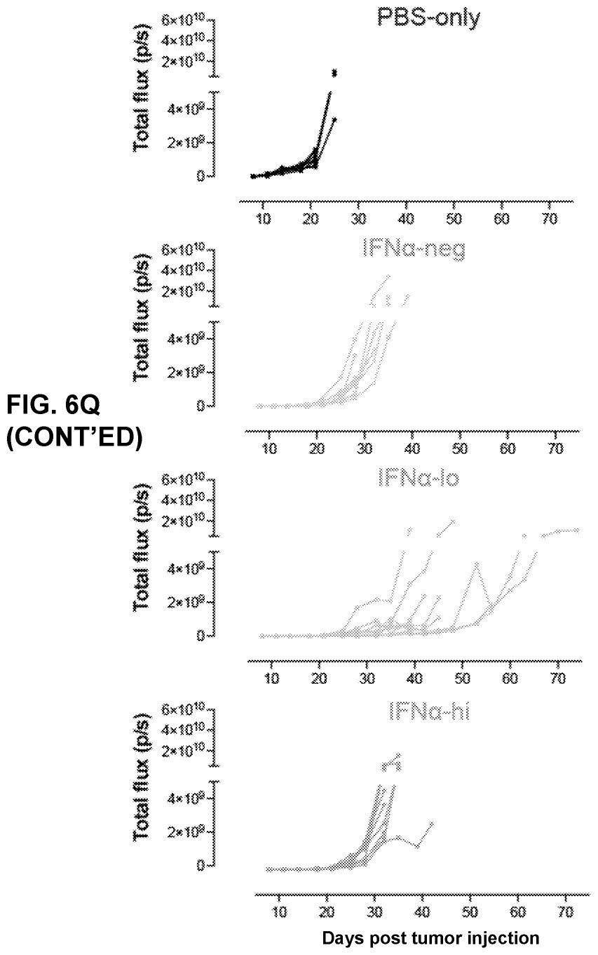

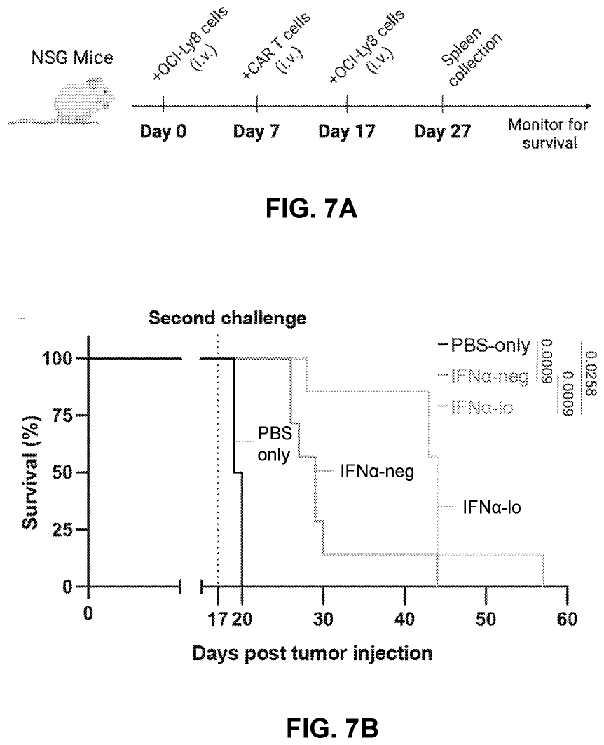

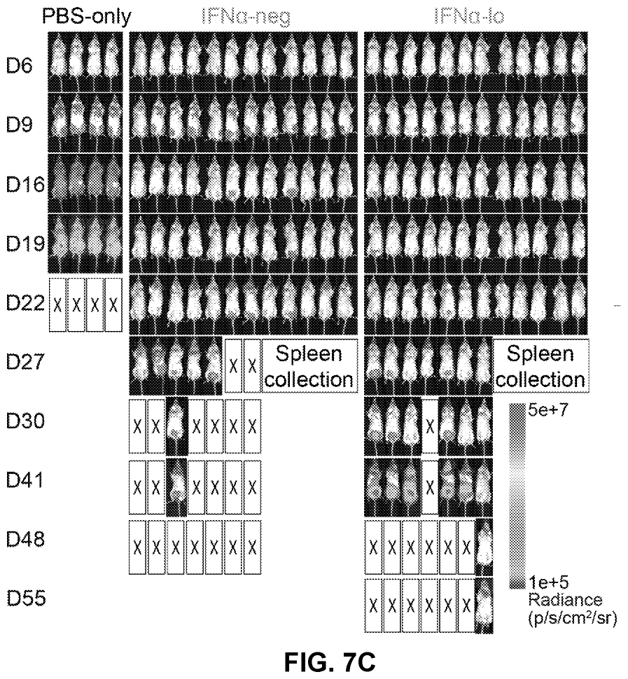

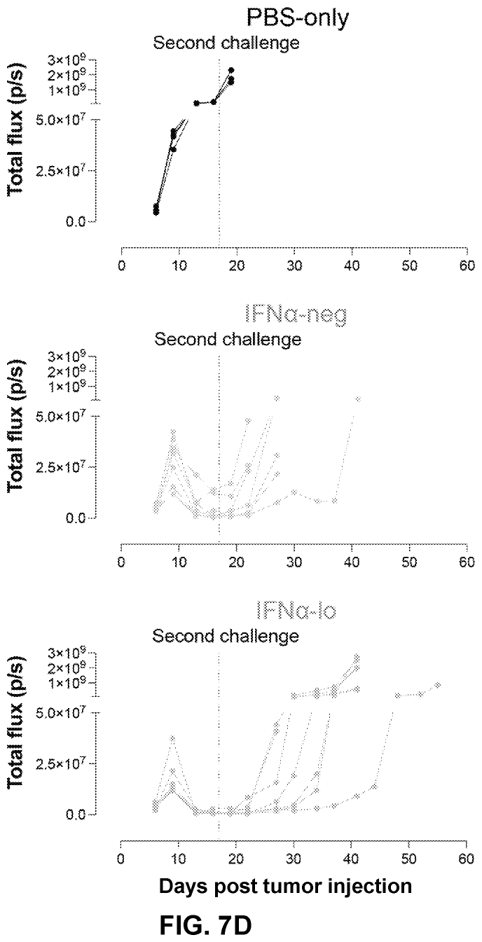

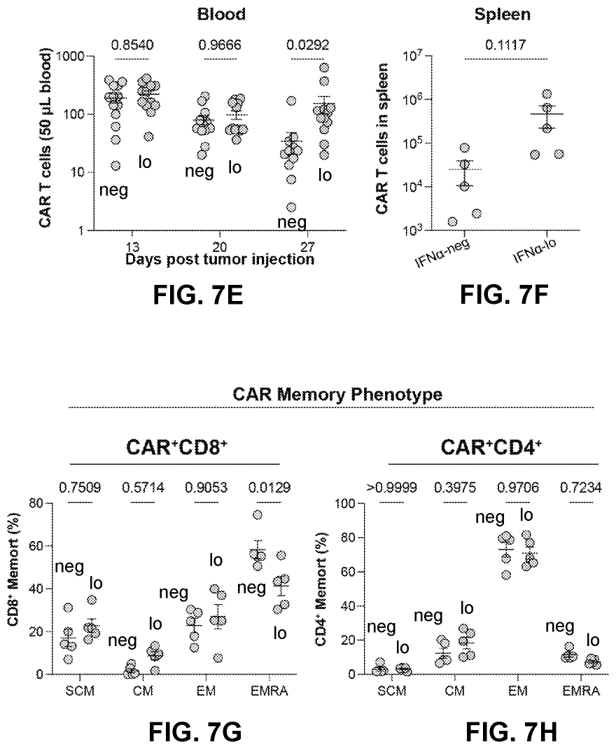

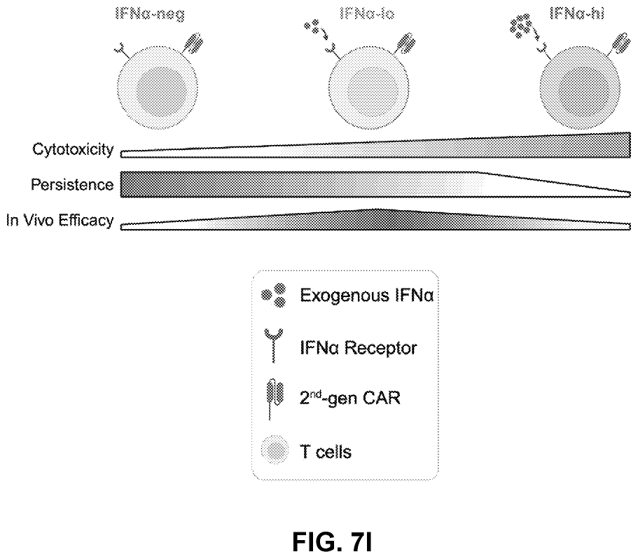

The population of IFN-enhanced CAR-T cells expresses a higher level of interferon-stimulated genes compared to a control population of CAR-T cells that is produced without co-culturing with the IFN receptor agonist. The population of IFN-enhanced CAR-T cells have several phenotypes that are advantageous in cancer therapy. For examples, the population IFN-enhanced CAR-T cells produces more IL-2, TNF, and/or IFNγ upon stimulation, exhibits higher expression of cytotoxic molecules compared to a control population of CAR-T cells that is produced without co-culturing with the IFN receptor agonist, which contribute to a higher efficiency in killing tumor cells. Meanwhile, the population of IFN-enhanced CAR-T cells has the same rate of persistence, as a control population (see FIGS. 6E and 6F for example); but exhibits a more durable efficacy. As used herein, the term “durable efficacy” refers the ability of CAR-T cells to maintain their therapeutic efficacy over time, see FIGS. 7A-7D, for example. The term “persistence” refers to the duration that CAR-T cells can survive and function in a patient's body or in a culture with tumor cells.

Pharmaceutical Composition and Method of Treatment with IFN-Enhanced CAR-T Cells

The present disclosure provides a pharmaceutical composition comprising a population of IFN-enhanced CAR-T cells. The population of IFN-enhanced CAR-T cells can be produced by the methods disclosed herein. The present disclosure also provides a method of treating cancer in a subject comprising providing a population of IFN-enhanced CAR-T cells to the subject or administering a therapeutically effective amount of the pharmaceutical composition comprising the population of IFN-enhanced CAR-T cells to the subject.

The pharmaceutical composition can be adminstered to the subject by intravenous infusion. The population of IFN-enhanced CAR-T cells can be produced from T cells isolated from PBMCs that are obtained from the same subject or from a donor.

The term “cancer” as used herein refers to an abnormal growth of cells which proliferate in an uncontrolled way and, in some cases, to metastasize (spread).

“Pharmaceutical composition” as used herein refers to a composition that includes a population of IFN-enhanced CAR-T cells, a pharmaceutically acceptable salt, carrier, a solvent, an adjuvant, and/or a diluent, or any combination thereof.

As used herein, the term “therapeutically effective amount” means at least the minimum dose of a population of IFN-enhanced CAR-T cells or a pharmaceutical composition comprising the population of IFN-enhanced CAR-T cells disclosed herein, necessary to achieve a desired therapeutic or prophylactic effect. Some embodiments include an amount sufficient to prevent or disrupt a disease process, deplete or ablate a cell population, or reduce the extent or duration of pathology. Some embodiments include a dose sufficient to reduce a symptom associated with the disease or condition being treated. An effective dosage or amount of a population of IFN-enhanced CAR-T cells or a pharmaceutical composition comprising the population of IFN-enhanced CAR-T cells disclosed herein can readily be determined by the person of ordinary skill in the art considering all criteria. In certain embodiments, the effective dosage or amount is 1×10⁵ to 5×10⁶ CAR T cells/kg.

The population of IFN-enhanced CAR-T cells can bind and kill tumor cells that express targets of the CAR such as CD19, CD20, CD22, or BCMA. Examples of cancer that can be treated with the disclosed methods include, but are not limited to, leukemia, multiple myeloma, diffuse large B cell lymphoma, acute myeloid leukemia, Mantle Cell lymphoma, follicular lymphoma, primary mediastinal B-cell lymphoma, acute lymphoblastic leukemia, or chronic lymphocytic leukemia. In some embodiments, the cancer comprises tumor cells that express CD19, such as diffuse large B cell lymphoma or leukemia.

“Treating” or “treatment,” as used herein, covers the treatment of a disorder, condition, or a disease described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder. For example, the terms “treating,” “treat,” or “treatment” refer to either preventing development or exacerbation of, providing symptomatic relief for, or curing a patient's disorder, condition, or disease.

As used herein, the terms “patient,” “subject,” and “individual” can be used interchangeably and refer to an animal. For example, the patient, subject, or individual can be a mammal, such as a human to be treated for a disorder, condition, or a disease.

For purposes of this invention, treating cancer includes, without limitation, alleviating one or more clinical indications, decreasing tumor growth or tumor cell proliferation, reducing the severity of one or more clinical indications of cancer condition, diminishing the extent of the condition, stabilizing the subject's disease state (i.e., not worsening), delay or slowing, halting, or reversing cancer progression, and bringing about partial or complete remission. Treating cancer also includes prolonging survival by days, weeks, months, or years as compared to prognosis if treated according to standard medical practice not incorporating or administering a population of IFN-enhanced CAR-T cells according to a method provided herein.

Improvement of the subject after receiving the treatment can be measured by increased overall survival, progression-free survival, radiographic response (e.g., as measured by PET imaging), or the subject's quality of life. In some embodiments, the overall survival of the subject is increased by at least two days after the treatment.

### EXAMPLES

The Examples that follow are illustrative of specific embodiments of the disclosure, and various uses thereof. They are set forth for explanatory purposes only and should not be construed as limiting the scope of the disclosure in any way.

### Methods

### Collection of Patient Biospecimens

Deidentified patient biospecimens were gathered in accordance with ethical guidelines under the institutional review board at the University of Chicago Medicine. Informed consent was obtained from all patients or their legal guardians prior to sample collection, in compliance with institutional and federal guidelines. All patients received commercially manufactured axi-cel (Yescarta®, Kite Pharma), generated using the same standardized GMP manufacturing protocol at a single external manufacturing facility. Patients were treated at UChicago Medicine between 2019 and 2021 and were selected based on the availability of a clearly defined clinical response, as determined by PET/CT imaging at 1 month post-CAR T-cell infusion, as well as the availability of residual infusion product cells for downstream analyses. Residual infusion product cells were collected from each patient's infusion bag, stored in CELLBANKER 1 (Amsbio, 11888), and subsequently cryopreserved in liquid-phase nitrogen for subsequent single-cell sequencing.

### Single-Cell RNA-Seq Data Generation

Cryopreserved biospecimens were thawed and washed with cold FACS buffer (PBS, 2% BSA, 0.05% sodium azide). Antibody-stained cells were conjugated with LIVE/DEAD Fixable Near-IR viability dye (Invitrogen, L34975) at 1:1000 dilution in PBS for 5 minutes at room temperature. Finally, cells were washed 3× in cold cell media (RPMI, 10% FBS) before sorting (BD Biosciences, FACSAria Fusion) for live cells. Sorted cells were partitioned into droplets for single-cell RNA-seq via Chromium Next GEM Single-Cell 5′Kit v2 (10× Genomics, 1000263). RNA-seq libraries were prepared according to manufacturer protocols. Libraries were quantified via the Qubit dsDNA HS Assay Kit (Invitrogen, Q32851), quality-checked for fragment sizes via high-sensitivity D5000 screentapes (Agilent, 5067-5592), pooled, and sequenced (Illumina, Novaseq-6000, RRID:SCR_016387 and Nextseq-550, RRID:SCR_016381).

### Single-Cell RNA-Seq Data Analysis

Alignment, filtering, counting, and demultiplexing of RNA-seq reads were performed using the Cell Ranger count (10× Genomics, version 7.1.0, RRID:SCR_017344). For alignment, the GRCh38 reference genome was appended with the CAR transgene sequence of axicabtagene ciloleucel.

Downstream analysis was performed using Seurat (Hao et al., 2021, Cell 184, 3573-3587) (version 4.3.0, RRID:SCR_016341). After converting count matrices and merging all Seurat objects, we filtered cells with >15% mitochondrial reads and 40,000 UMIs to remove debris, doublets, and dead cells. The matrix was normalized using NormalizeData with default options. The top 5,000 variable genes found by FindVariableFeatures with the default “vst” method were taken as variable genes. Data were centered and scaled using ScaleData, with additional regression against (1) the percent of mitochondrial RNA content and (2) difference in cell cycle S-phase and G2M-phase scores. Scaled data were then used for principal component analysis (PCA) using RunPCA. Data harmonization to remove patient-specific effects was performed on the principal components using Harmony (Korsunsky et al., 2016, Nat. Methods 16, 1289-1296) (version 0.1.1, RRID:SCR_018809) through RunHarmony. Top 50 harmony components were used for Uniform Manifold and Projection (UMAP) dimensionality reduction via RunUMAP. The same harmony components were used to construct the shared nearest neighbor (SNN) graph using FindNeighbors, which was then partitioned to identify clusters using FindClusters with the “original Louvain” algorithm.

Differentially expressed genes were determined using FindMarkers in Seurat package with default parameters. Gene set enrichment analysis and pathway enrichment analysis were carried out using clusterProfiler (Wu et al., 2021, Innovation (Camb). 2, 100141) (version 4.6.2, RRID:SCR_016884) based on pathways from the msigdbr database's built-in msigdbr²⁸ package (version 7.5.1, RRID:SCR_022870). Gene module scores were calculated for each cell using AddModuleScore in Seurat for msigdbr or customized gene sets.

### Cell Lines

The OCI-Ly8 (RRID:CVCL_8803) cell line was obtained from Dr. James L. LaBelle's lab (University of Chicago), the VAL cell line (RRID:CVCL_1819) was obtained from Dr. Justin P. Kline's lab (University of Chicago), the NALM6 cell line was obtained from ATCC (CRL-3273™, RRID:CVCL_0092), and the Lenti-X 293T packaging cell line was obtained from Takara (632180, RRID:CVCL_4401). All cell lines were confirmed to be mycoplasma-negative using the Universal Mycoplasma Detection Kit (ATCC, 30-1012k), with the most recent testing performed in November 2025. All cell lines were used for experiments within three passages after thawing.

### Lentiviral Production and Transduction

CD28- or 4-1BB-costimulated anti-CD19 human CARs were designed based on the sequences of axi-cel or liso-cel. The axi-cel sequence was obtained from a previously published sequencing study (Cao et al., 2025, Nat. Commun. 16, 4205), while the liso-cel sequence was retrieved from the Global Substance Registration System database (UNII: 7K2YOJ14×0). A GFP-tagged, CD28-costimulated or 4-1BB-costimulated, anti-CD19 human CAR was cloned onto the pHR vector backbone to constitute the transfer plasmid. Plasmids were transfected into the Lenti-X 293T packaging cell line (Takara, 632180, RRID:CVCL_4401) for lentiviral production. In brief, Lenti-X 293T cells were seeded and grown overnight to ~90% confluence. Subsequently, transfer, packaging (psPAX2, Addgene, #12260, RRID:Addgene_12260), and envelope (pMD2.G, Addgene, #12259, RRID:Addgene_12259) plasmids were co-transfected into Lenti-X 293T cells via Lipofectamine 3000 (Invitrogen, L3000001). After 72 hours, the viral supernatant was collected and stored at −80° C. until transduction. During transduction, lentiviruses were added to human primary T cells along with protamine sulfate (Millipore Sigma, P3369-10G) to a final concentration of 10 μg/mL. Cells were spinoculated at 800×g for 60 minutes at room temperature. All cell lines were routinely tested for Mycoplasma contamination every 4-6 weeks using a PCR-based assay.

### CAR T-Cell Production and Culturing

PBMC samples were collected from healthy donors and DLBCL patients under an Institutional Review Board approved protocol. Cryopreserved human PBMCs were thawed in warm T-cell media (RPMI supplemented with 10% FBS, 1% penicillin/streptomycin, 2 mM L-glutamine, and 50 μM 2-mercaptoethanol). Subsequently, PBMCs were cultured for 3 hours in complete T-cell media (T-cell media supplemented with 50 units/mL of IL-2) in order to allow monocytes to adhere to the culture flask. The non-adhered cells were transferred to a new culture flask. Cells were counted via the LUNA-FL cell counter. T-cell percentage (% CD3⁺) was calculated by flow cytometry.

T cells were seeded in complete T-cell media at 0.75×10⁶/mL. CD3/CD28 Dynabeads (Gibco, 11161D) were washed and added at 1:1 ratio. After overnight activation, T cells were transduced with CAR lentiviruses (MOI of 5). The media was refreshed after 24 hours with complete T-cell media dosed with recombinant human IFN-α2 (BioLegend, 592702). T cells were subsequently expanded, and media was refreshed every 2 days.

### Quantitative Reverse Transcription PCR

CAR T cells were treated under IFN-I culturing conditions for 24 hours. Then, cells were collected, and stained for viability (Invitrogen, L34975). Live CAR T cells were sorted (BD Biosciences, FACSAria Fusion). Total RNA was extracted using the Monarch Total RNA Miniprep Kit (NEB, T2010S), according to manufacturer protocols. RNA was quantified via the Qubit RNA HS Assay Kit (Invitrogen, Q32852). cDNA was synthesized using the GoScript Reverse Transcription System (Promega, A5000).

Quantitative PCR was performed using Taqman Gene Expression Assay (ThermoFisher Scientific, 4331182) and Applied Biosystems TaqMan Universal PCR Master Mix (ThermoFisher Scientific, 4304437), on LightCycler® 96 Instrument (Roche). The cycling conditions were as follows: UNG incubation at 50° C. for 2 minutes, initial denaturation at 95° C. for 10 minutes, followed by 40 cycles of denaturation at 95° C. for 15 seconds and annealing/extension at 60° C. for 60 seconds. Each sample was run in triplicate.

The specific Taqman Gene Expression Assay used were as follows: ACTB (Hs01060665_g1), IRF7 (Hs01014809_g1), MX1 (Hs00895608_m1), and ISG15 (Hs01921425_s1). Relative gene expression levels were calculated using the 2\hat{ }-ΔΔCt method, with ACTB serving as the internal control. Statistical analysis was performed using one-way ANOVA followed by a Tukey's post hoc test for significance.

### In Vitro Co-Culture Immunoassays

For all co-culture immunoassays, CAR T cells (E) and luciferized OCI-Ly8 cells (T, RRID:CVCL_8803) were seeded under a fixed E/T ratio. To measure in vitro cytotoxicity, co-cultures were established with E/T ratio of 1:1. After 6 hours, One-Glo Luciferase substrate (Promega, E6110) was added to each well. Residual tumor cells were quantified via luciferase activity. As a positive control, target cells were directly lysed with 1% NP-40 (Abcam, ab142227). To measure cytotoxic molecule expression, co-cultures were established with E/T ratio of 1:1 with 1× brefeldin A (BioLegend, 420601). After 4 hours, cells were collected for intracellular staining and flow cytometry.

To measure cytokine secretion, co-cultures were established with E/T ratio of 1:1. After 24 hours, the culture supernatant was collected and stored. Secreted cytokines were quantified using LEGENDplex immunoassays per manufacturer's protocol (BioLegend). To measure in vitro persistence, co-cultures were established with E/T ratio of 1:5. Every three days, cells were counted using Precision Count Beads (BioLegend, 424902) and measured for apoptosis on flow cytometry. Cell media was then refreshed.

### Flow Cytometry

Cells were washed with cold FACS buffer (PBS, 2% BSA, 0.05% sodium azide). Fc receptors were blocked by incubation with Human TruStain FcX (BioLegend, 422301) at 1:50 dilution for 5 minutes at 4° C. Then, cells were incubated for 30 minutes at 4° C. in the dark with an antibody staining solution for surface antigen staining. Antibodies were used according to manufacturer recommendations. After staining, cells were incubated briefly either with live/dead staining solution or apoptosis staining solution for 10 minutes at room temperature. Then, cells were either washed and resuspended in FACS buffer or resuspended in Annexin V Binding Buffer (BioLegend, 422201). Cells were ready for analysis by flow cytometry or intracellular staining.

For intracellular staining, cells stained for surface antigens were subject to the BD Fixation/Permeabilization Kit (BD Biosciences, 554714) for cytotoxic molecule staining or True-Nuclear™ Transcription Factor Buffer Set (BioLegend, 424401) for IRF7 staining, following manufacturer recommendations. In brief, Cells were fixed in fixation buffer, washed 3× in permeabilization buffer, and incubated for 60 minutes at 4° C. in the dark with a staining solution containing antibodies and permeabilization buffer. After staining, cells were washed 2× in permeabilization buffer, 1× in FACS buffer, and resuspended in FACS buffer for analysis by flow cytometry.

For Ki-67 staining, cells stained for surface antigens were washed 2× in PBS, fixed in prechilled 70% ethanol overnight, washed 2× in FACS buffer, and incubated for 30 minutes at 4° C. in the dark with Ki-67 antibody staining solution. After staining, cells were washed 2× in FACS buffer, and resuspended in FACS buffer for analysis by flow cytometry.

Flow cytometry was performed on LSRFortessa (BD Biosciences, RRID:SCR_018655) or Aurora (Cytek Biosciences, RRID:SCR_019826). Data were analyzed using FlowJo software (version 10.10.0, RRID:SCR_008520).

### Antibodies

All antibodies were used according to manufacturer recommendations. APC-anti-IFNAR2 (10359-MM07T-A) was from Sino Biological. All other antibodies originate from BioLegend. For surface staining, AF647-anti-CD3 (clone: UCHT1, 300416, RRID:AB_389332), BV421-anti-CD3 (clone: UCHT1, 300434, RRID:AB_10962690), BV510-anti-CD4 (clone SK3, 344633, RRID:AB_2566016), PerCP/Cy5.5-anti-CD8a (clone SKi, 344709, RRID:AB_2044009), PE-anti-CD45RA (clone HI100, 304107, RRID:AB_314411), BV421-anti-CD45RO (clone UCHL1, 304223, RRID:AB_10898323), AF647-anti-CCR7 (clone G043H7, 353217, RRID:AB_10913812), BV421-anti-PD-1 (clone EH12.2H7, 329919, RRID:AB_10900818), PE/Cy7-anti-LAG-3 (clone 11C3C65, 369309, RRID:AB_2629752), PE/Dazzle™ 594-anti-TIGIT (clone A15153G, 372715, RRID:AB_2632930), PE/Cy7-anti-CCR7 (clone G043H7, 353225, RRID:AB_11125576), and BV785-anti-CD62L (clone DREG-56, 304829, RRID:AB_2629516) were used. For intracellular staining, PE/Cy7-anti-granzyme B (clone QA16A02, 372213, RRID:AB_2728380), PE-anti-perforin (clone B-D48, 353303, RRID:AB_10915476), and PE-anti-IRF7 (clone 12G9A36, 656003, RRID:AB_2563006) were used. For live/dead staining, Zombie NIR Fixable Viability Kit (423106) was used in PBS. For apoptosis staining, BV421-Annexin V (640923, RRID:AB_2893503) or PE/Cy7-Annexin V (640950) was diluted in Annexin V Binding Buffer (422201). For Ki-67 staining, PE-anti-Ki-67 (clone Ki-67, 350503, RRID:AB_10660818) was used.

### Lymphoma Xenograft Mouse Model

Immunocompromised NOD-scid IL2Rgamma^{null} (NSG, Strain #005557) mice were purchased from JAX and maintained at the animal resource center (ARC) of the University of Chicago in accordance with institutional guidelines. Mice were monitored daily. Care and experimental procedures were carried out in line with the Institutional Animal Care and Use Committee (IACUC) approved protocols of the Animal Care Center at the University of Chicago.

For the standard xenograft models, 5×10⁶ OCI-Ly8-Chili-luc or NALM6-Chili-luc cells in 100 μL PBS were intravenously injected into 8-12-week-old NSG mice. Mice were randomized prior to CAR T-cell injection. 5×10⁵ or 1×10⁶ CAR T cells were intravenously injected 1 week later for the OCI-Ly8 model or 4 days later for the NALM6 model. For the OCI-Ly8 rechallenge model, 3×10⁶ OCI-Ly8-Chili-luc cells were intravenously injected on day 0, followed by randomization and intravenous injection of 3×10⁵ CAR T cells 1 week later. Mice were then rechallenged with an additional 3×10⁶ OCI-Ly8-Chili-luc cells intravenously 10 days after CAR T-cell infusion. Lymphoma progression was monitored using either IVIS Spectrum (PerkinElmer) or Lago X (Spectral Instruments Imaging) following the Intraperitoneal injection of D-luciferin potassium (PerkinElmer, 122799). Animals were anesthetized at 2% isoflurane throughout acquisition. Bioluminescence was quantified with Aura software (Spectral Instruments Imaging). Euthanasia was conducted via CO₂ overdose followed by cervical dislocation upon manifestation of paralysis, impaired mobility, or poor body condition (BCS<2). Group size were determined based on prior experience with the xenograft model and expected treatment effect sizes.

Blood was collected by cheek bleeding using a 4-mm lancet (Fisher Scientific, NC9922361), and 50 μL was subjected to red blood cell (RBC) lysis using RBC lysis buffer (eBioscience, 00-4300-54). Lysis was quenched with FACS buffer, and cells were washed once with FACS buffer before proceeding to flow staining. Spleens were collected by surgery and gently ground through a 40-μm cell strainer, followed by RBC lysis using RBC lysis buffer (eBioscience, 00-4300-54). After stopping the lysis with RPMI supplemented with 10% FBS, cells were washed once more with RPMI+10% FBS before being subjected to flow staining.

### Statistical Analysis

Statistical tests were performed on R (version 4.2.1, RRID:SCR 001905) or GraphPad Prism 10 (version 10.1.1, RRID:SCR_002798). Comparisons between two groups were performed using Wilcoxon Rank-Sum test. Comparisons between more than two groups were performed by either one-way or two-way ANOVA followed by Tukey correction for multiple comparisons. Survival data were analyzed using the Log-Rank (Mantel-Cox) test. To assess correlation, Spearman's rank correlation coefficient was calculated, as indicated.

### Data Availability

All single-cell data of discovery dataset have been deposited in the Gene Expression Omnibus (GEO) and are available with the accession code GSE207192 (complete responders)²⁹ and GSE283351 (patients with progressive disease). Data of validation dataset is available on GEO at GSE197268.

### Code Availability

Codes supporting the analyses performed in this study are available at github.com/ertingtang/IFN-I-CART.

### Example 1: Enrichment of IFN-I Signaling in Infusion Products of Complete Responders

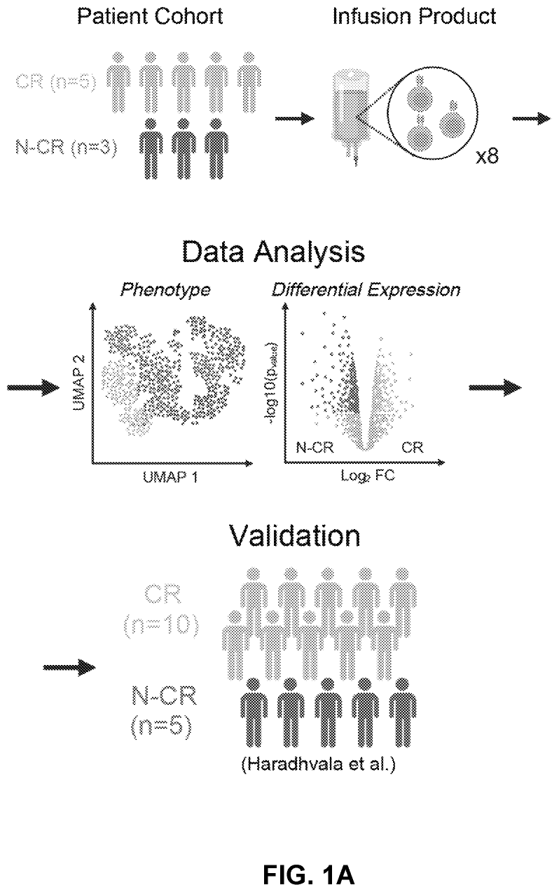

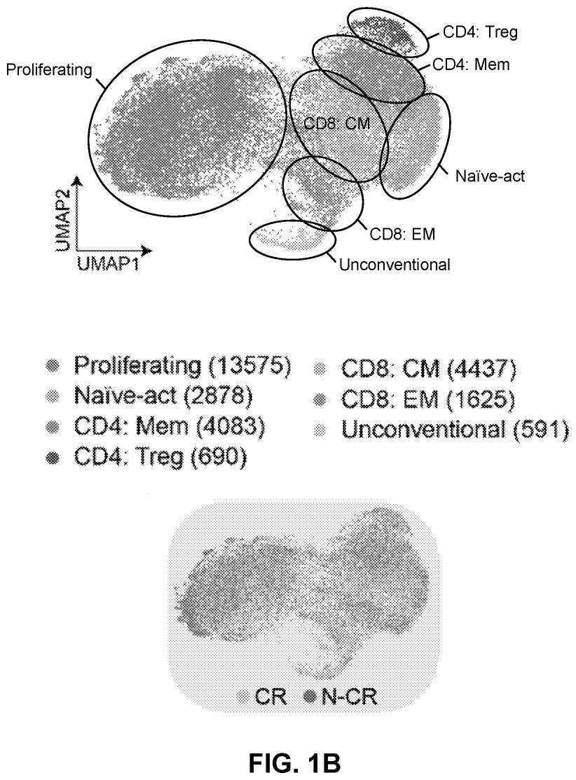

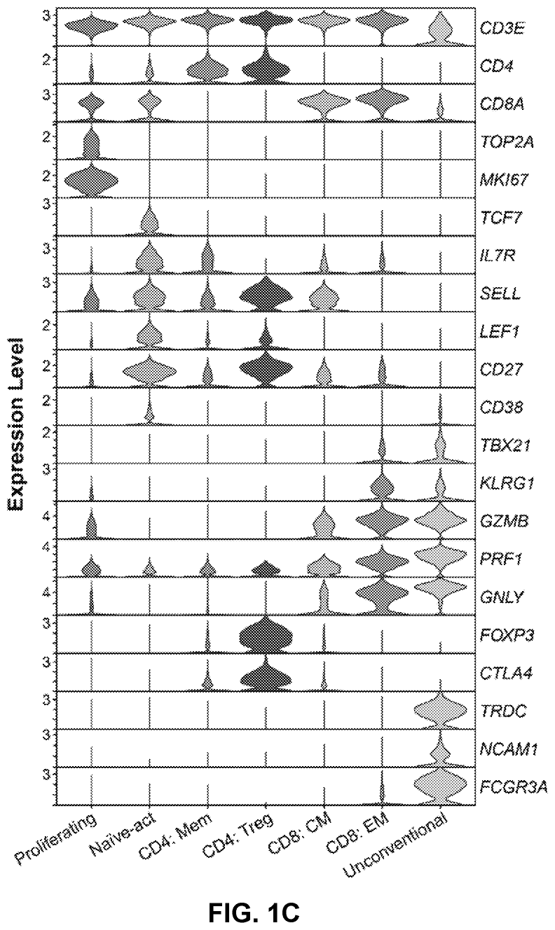

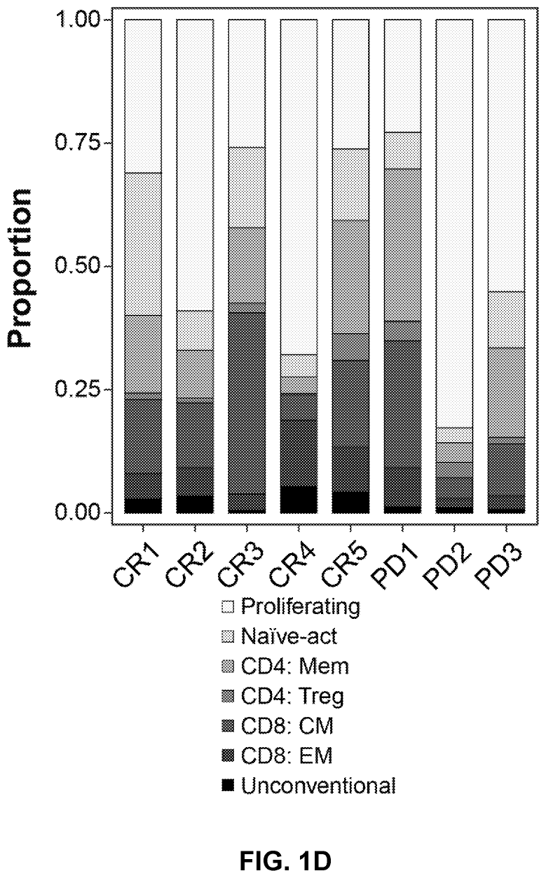

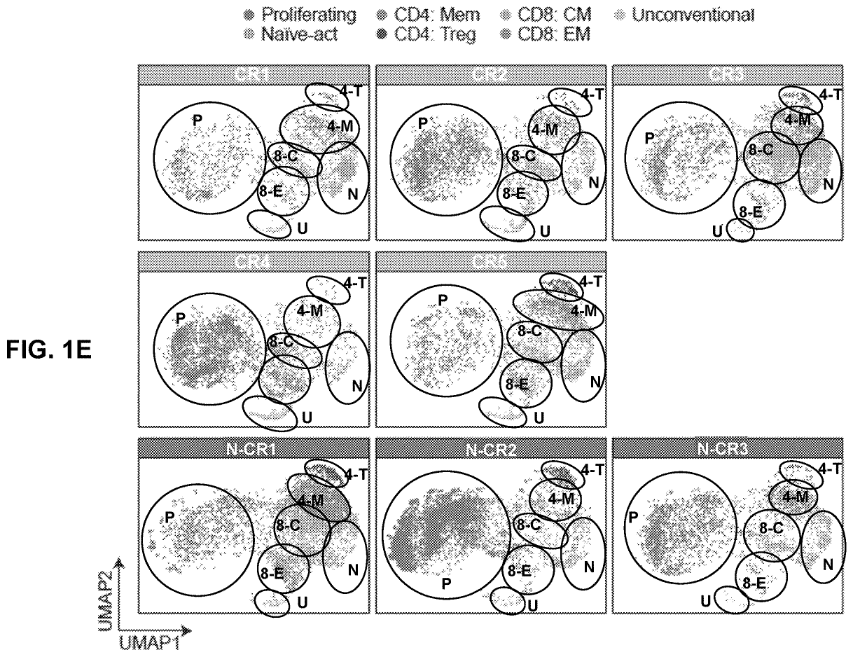

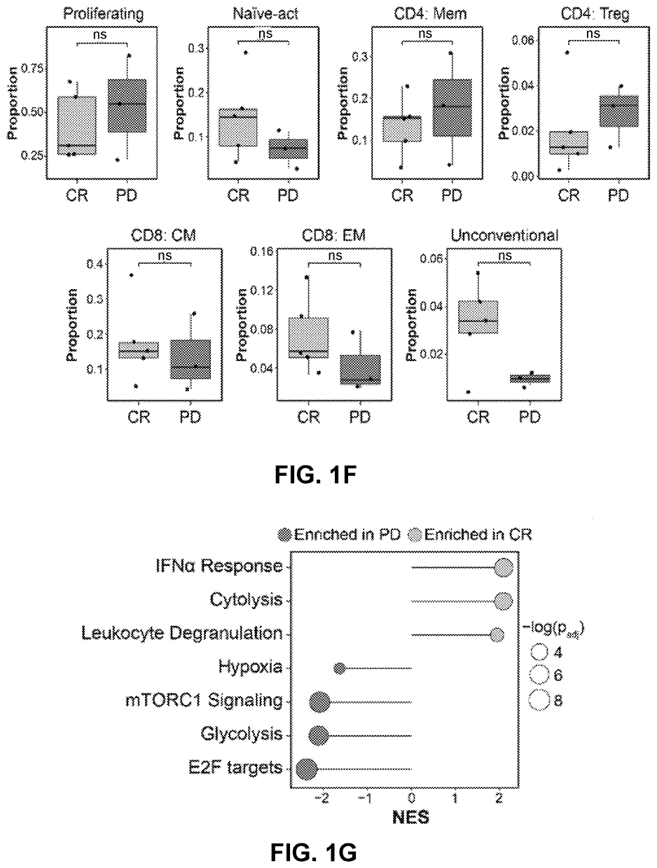

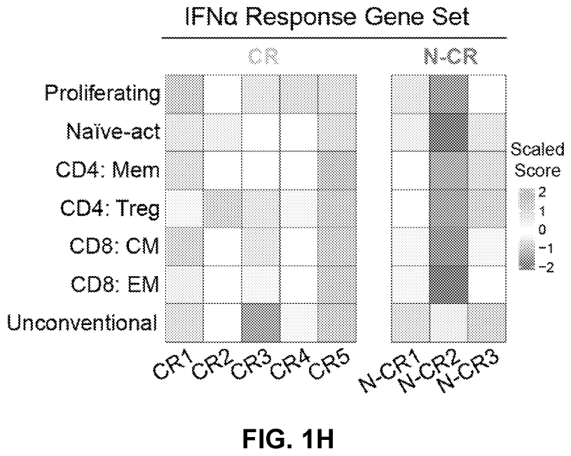

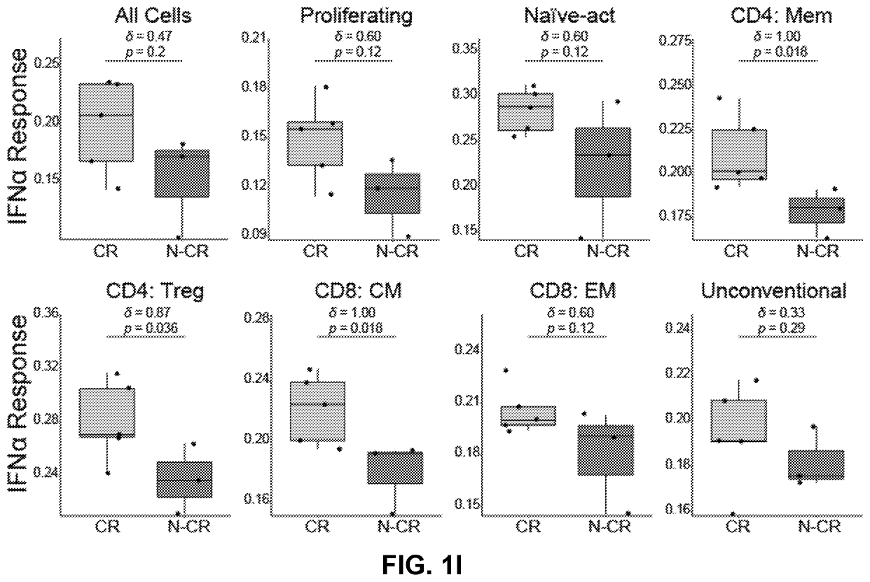

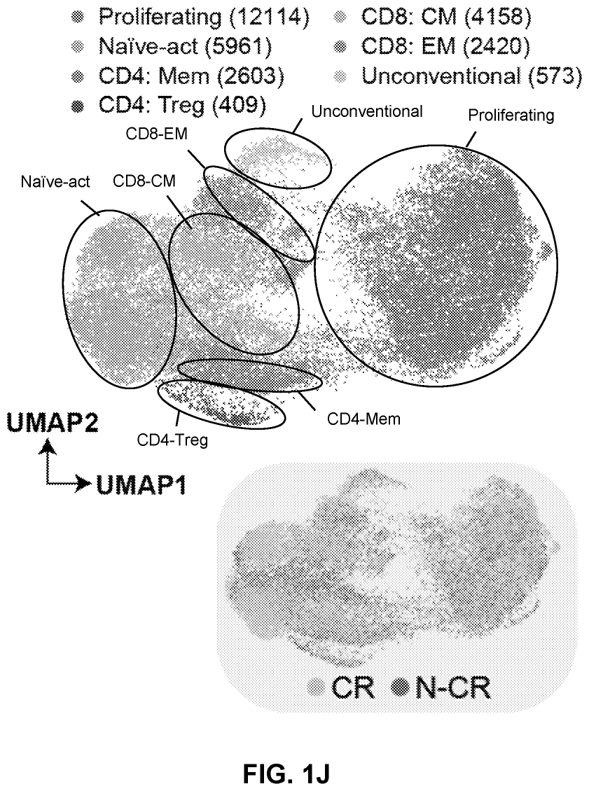

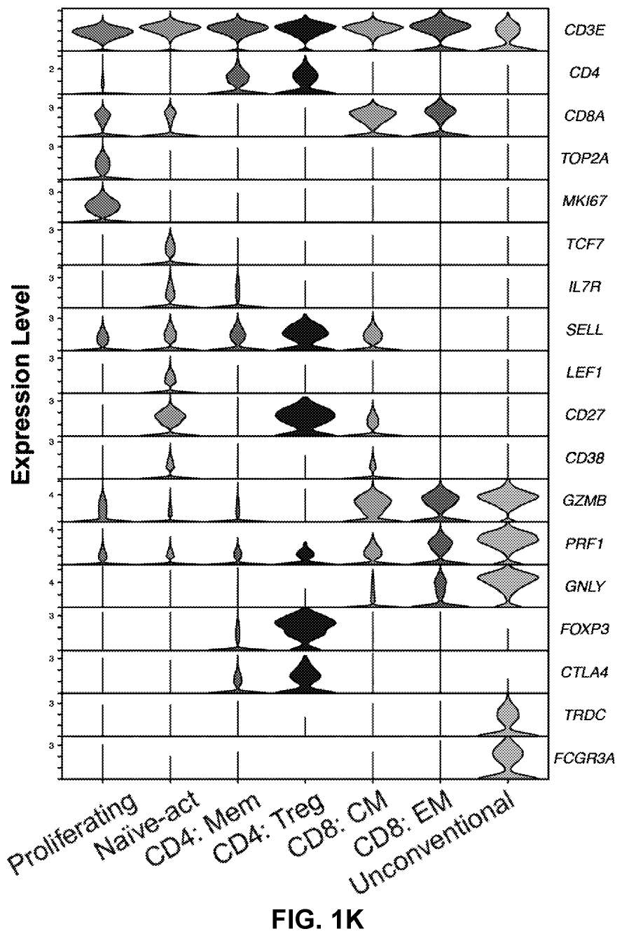

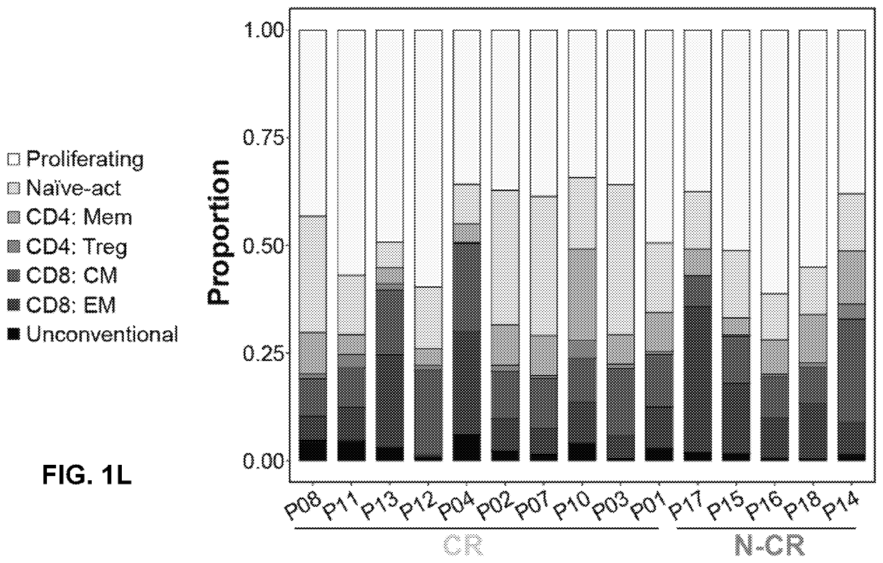

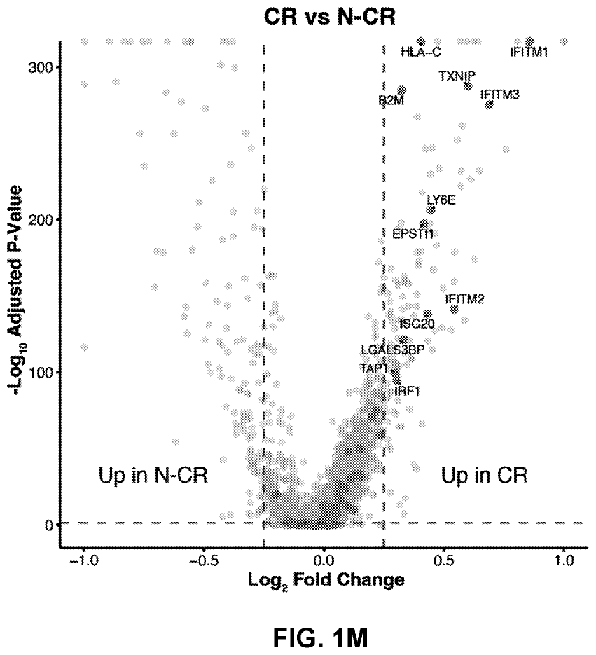

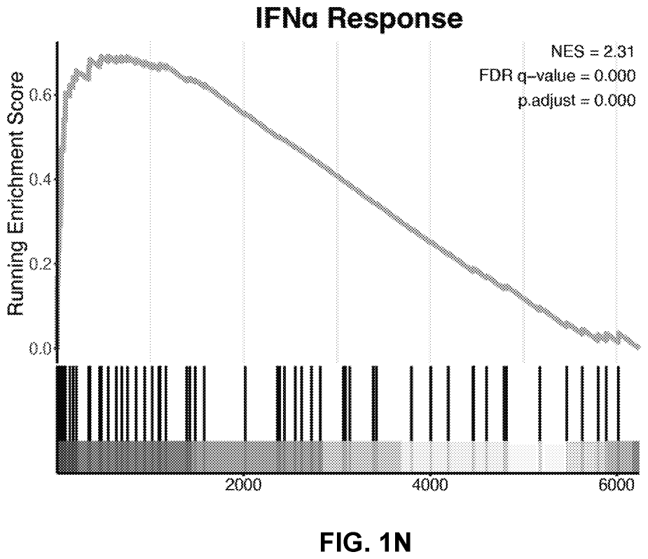

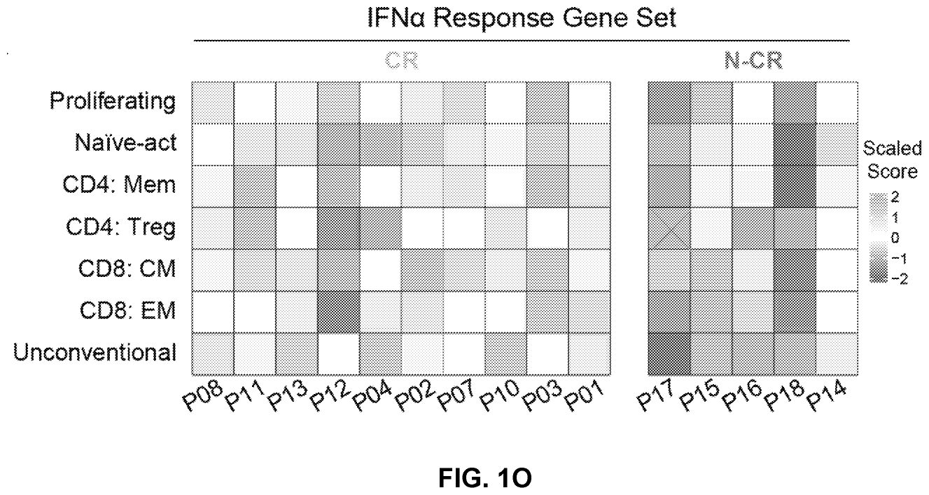

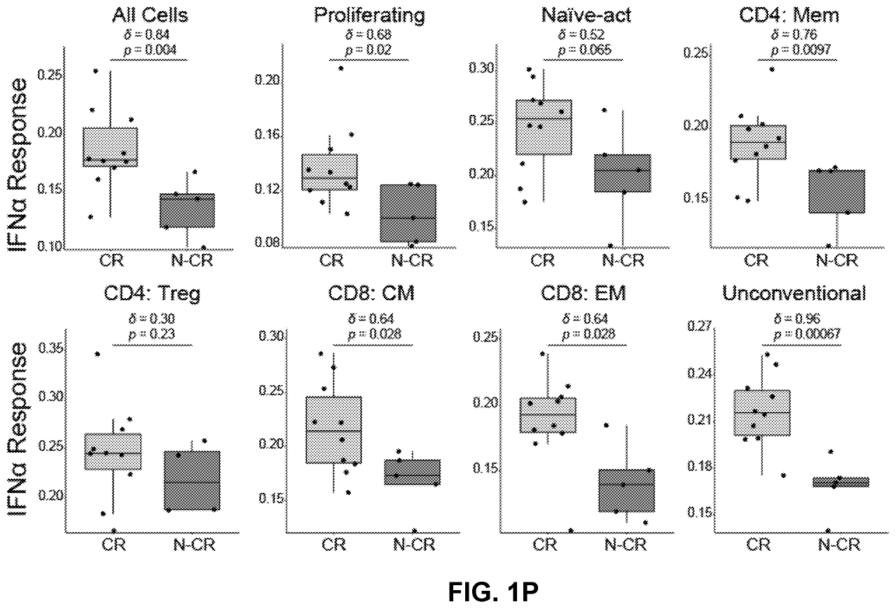

To uncover determinants of therapeutic efficacy, infusion products from 8 relapsed/refractory diffuse large B-cell lymphoma (r/r DLBCL) patients treated with CD28-costimulated CD19-directed CAR T cell therapy (axicabtagene ciloleucel, “axi-cel”) at the University of Chicago Medicine were profiled using single-cell sequencing. The cohort included 5 patients who achieved a complete response (CR) at one month post-infusion and another 3 patients who exhibited progressive disease (N-CR) (FIG. 1A and Table 1). After removing low-quality cells, we obtained 27,879 cells distributed among 7 T-cell clusters (FIG. 1B) based on gene expression (FIG. 1C). No cluster was patient specific (FIG. 1D and FIG. 1E). The two CD8+ clusters were annotated as central memory (CM, SELL⁺IL7R⁺TBX21⁻KLRG1⁻) or effector memory (EM, SELL⁻IL7R⁺TBX21⁺KLRG1⁺) T cells. The two CD4+ clusters were annotated as memory (Mem, FOXP3^{lo}IL7R⁺CTLA4^{lo}) or regulatory (Treg, FOXP3^{hi}IL7R⁻CTLA4^{hi}) T cells. The remaining three clusters consisted of naïve-activated (SELL⁺IL7R⁺ TCF7⁺CD38⁺), unconventional (TRDC⁺NCAM1⁺) as a mixture of γδ and natural killer phenotypes, and proliferating (MKI67⁺ TOP2A⁺) T cells. No discernable composition differences were found between CR and N-CR patients (FIG. 1B, FIG. 1D, and FIG. 1F).

Gene set enrichment analysis revealed distinct differences between CR and N-CR T cells. CR T cells were enriched for signatures of type I interferon signaling (“IFNα response”), cytolysis, and leukocyte degranulation, whereas N-CR T cells were enriched for signatures of cell growth, hypoxia, and glycolysis (FIG. 1G). Notably, the CR-specific IFNα response signature was enriched in CR T cells across T-cell clusters and individual patients, with Cliff's δ indicating moderate to strong differences between CR and N-CR groups (FIG. 1H and FIG. 1I).

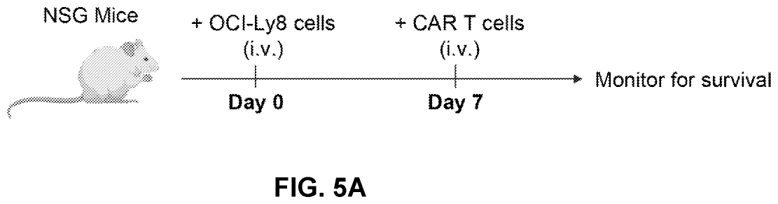

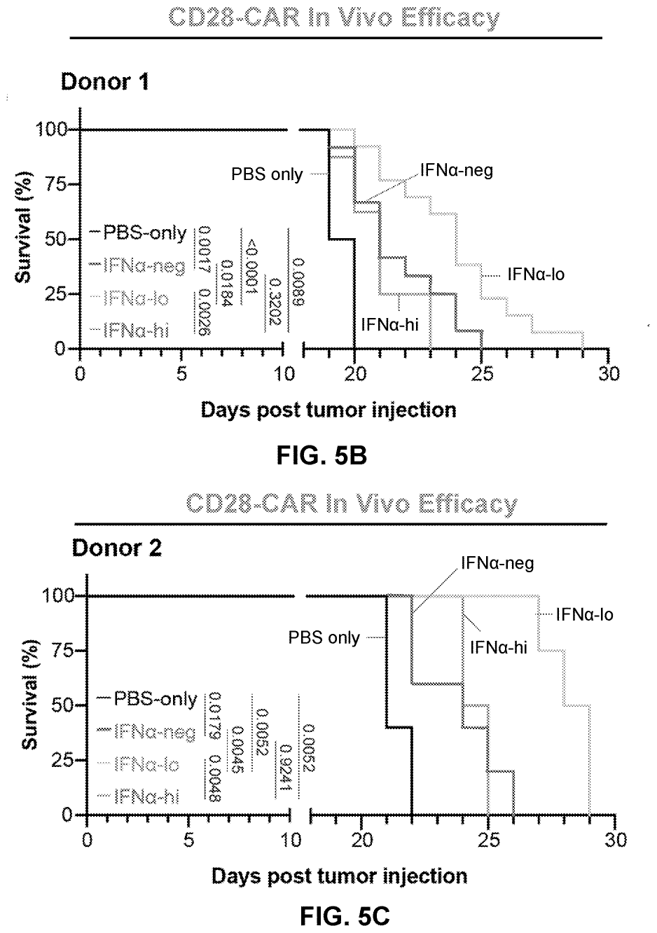

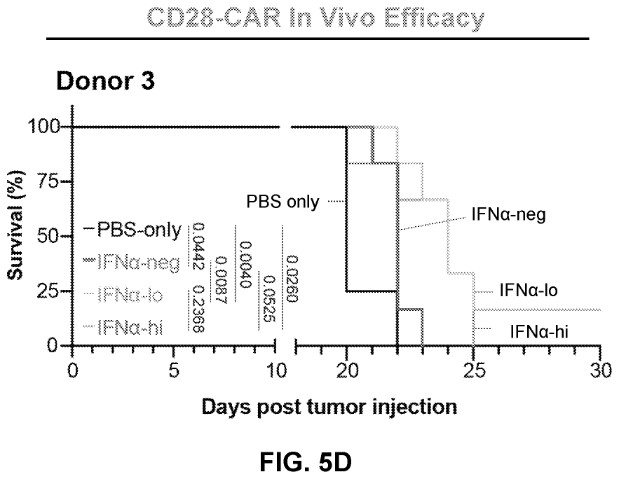

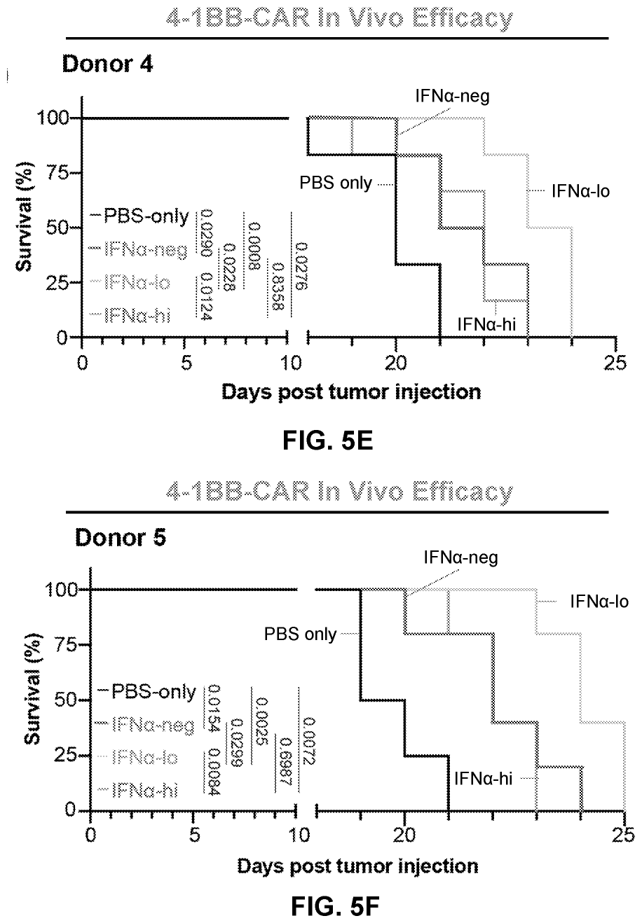

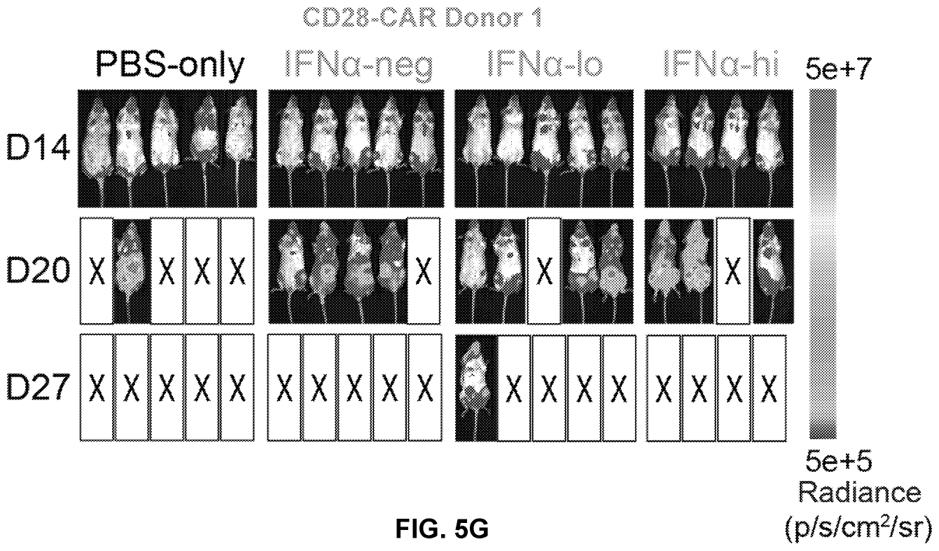

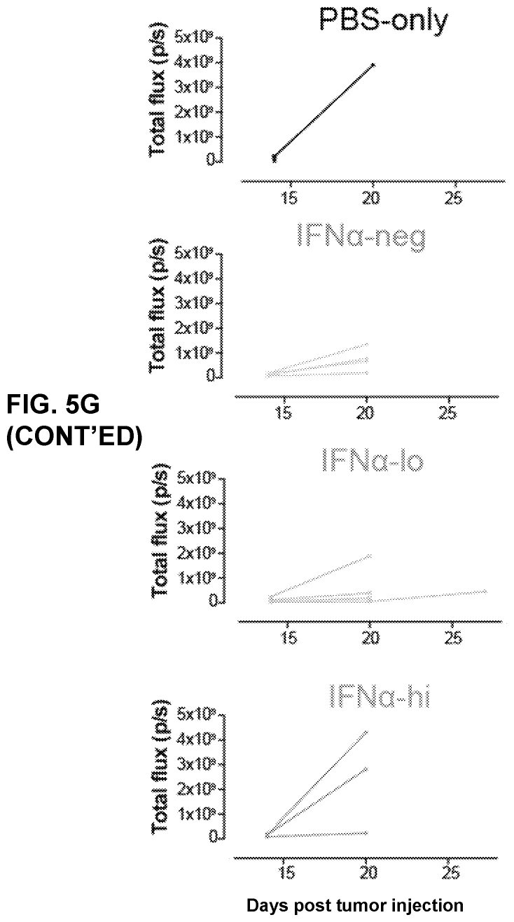

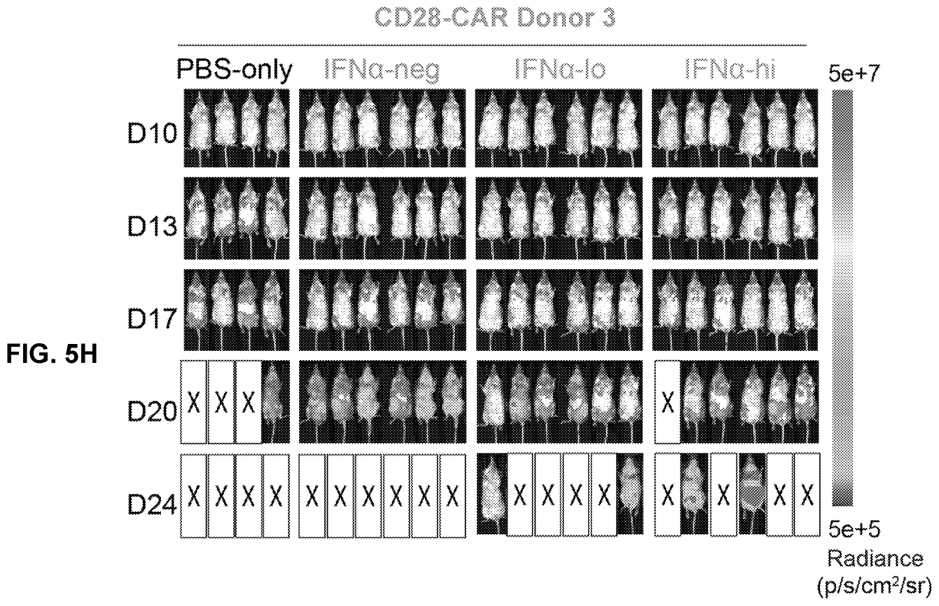

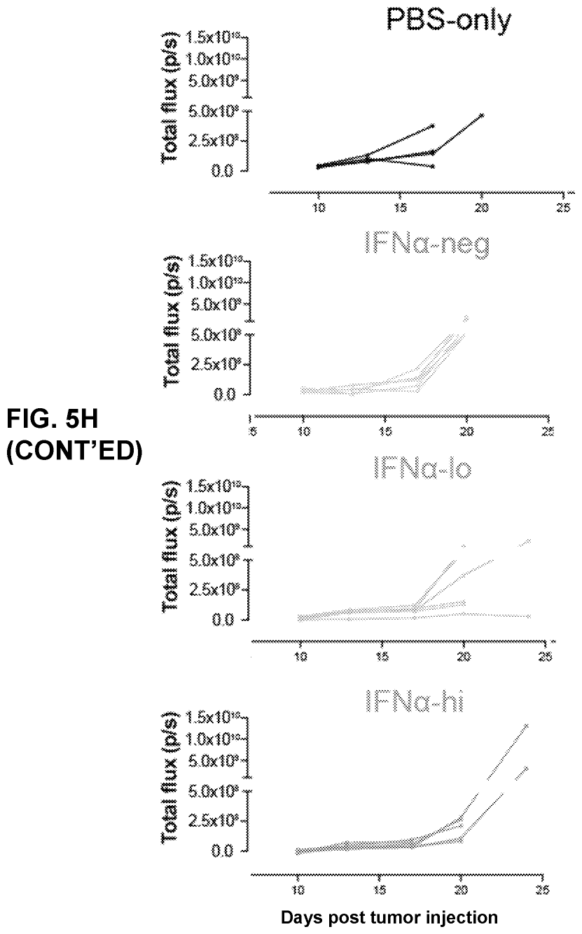

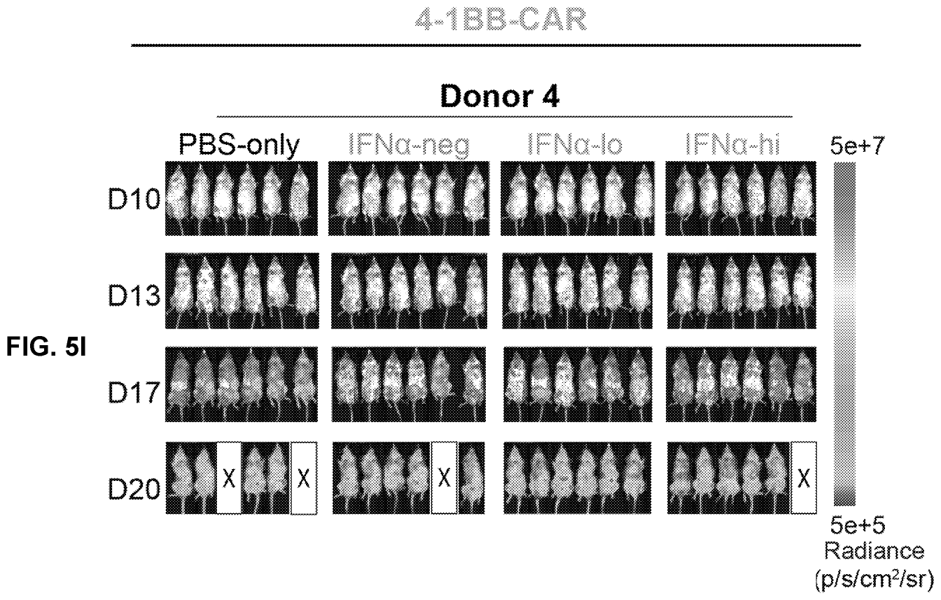

To validate the discovery of CR-specific IFN-I signaling signatures, the findings were tested using an independent and published single-cell transcriptomic dataset from Maus et ae. (n=15 patients with lymphoma who received axi-cel, FIG. 7A) (Haradhvala et al., 2022, Nat. Med 28, 1848-1859). This cohort included 10 patients who achieved a complete response (CR) and 5 who did not (N-CR), as assessed one-month post-infusion. After filtering out low-quality cells, performing dimensionality reduction, and clustering, the same 7 T-cell clusters identified in the previous dataset were uncovered (FIG. UJ) based on gene expression (FIG. 1K). No cluster was patient specific (FIG. 5L). Notably, expression of ISGs (including ISG2, IFITM1/3) was upregulated in IP of CR (FIG. 1M). Most importantly, IFNα response signatures was enriched in IPs of CR (FIG. 1N). The CR-specific enrichment of IFNα response signature was consistently observed across all clusters except Tregs and across patients, again with Cliff s δ indicating moderate to strong differences between CR and N-CR groups (FIG. 1O and FIG. 1P).

Taken together, findings from the discovery and validation datasets strongly support an association between IFN-I signaling and improved CAR T-cell clinical efficacy.

TABLE 1
Patient characteristics and relevant clinical data.

| Characteristics | CR-1 | CR-2 | CR-3 | CR-4 | CR-5 | N-CR-1 | N-CR-2 | N-CR-3 |
|---|---|---|---|---|---|---|---|---|
| Age Decade, y | 50s | 70s | 50s | 40s | 60s | 70s | 60s | 50s |
| Sex | Female | Male | Female | Male | Male | Male | Female | Male |
| Disease stage | I | II | II | IV | IV | IV | III | I |
| No. of prior | 4 | 2 | 2 | 5 | 5 | 3 | 3 | 3 |
| therapies | | | | | | | | |
| Prior lines of | Radiation | R-CHOP | DA- | R-CHOP | R-CHOP | BR | BR | R-CHOP |
| therapy | | | EPOCH | | | | | |
| | | | w/IT | | | | | |
| | | | MTX | | | | | |
| | R-CHOP | R-ICE | ICE | R-ICE | Benda- | DA-R- | ICE | DA-R- |
| | | | | | obinutuzumab | EPOCH | | EPOCH |
| | R-ICE | | | BEAM + | R-ICE | Hu5F9- | GEMOX | R-ICE |
| | | | | ASCT | | G4 + | | |
| | | | | | | rituximab | | |
| | BEAM + | | | FCR + | Hu5F9- | | | |
| | ASCT | | | AlloSCT | G4 + | | | |
| | | | | | rituximab | | | |
| | | | | R- | R- | | | |
| | | | | GEMOX | GEMOX | | | |
| Disease status | Relapsed | Relapsed | Primary | Relapsed | Relapsed, | Primary | Relapsed, | Primary |
| | | | refractory | | then | refractory | then | refractory |
| | | | | | refractory | | refractory | |

Baseline
(Ref. range)

| | | | | | | | | |
|---|---|---|---|---|---|---|---|---|
| LDH, U/L | 186 | 165 | 312 | 203 | 311 | 332 | 336 | 260 |
| (116-245 U/L) | | | | | | | | |
| CRP, mg/dL | 0.6 | 0 | 3.5 | 0.3 | ND | 0.6 | 0 | 0.2 |
| (<0.5 mg/dL) | | | | | | | | |
| Ferritin, ng/mL | 215 | 110 | 576 | 544 | ND | 64 | 124 | 294 |
| (20-300 ng/mL) | | | | | | | | |
| ECOG PS | 0 | 1 | 2 | 1 | 1 | 0 | 1 | 1 |
| Bridging therapy | No | No | Yes | Yes | No | No | Yes | No |
| Maximum Grade | 1 | 2 | 2 | 1 | 1 | 1 | 1 | 0 |
| CRS | | | | | | | | |
| Maximum Grade | 4 | 2 | 3 | 1 | 3 | 0 | 0 | 0 |
| ICANS | | | | | | | | |
| Tocilizumab or | Steroids | Both | Both | Tocilizumab | Both | Neither | Tocilizumab | Neither |
| steroids | | | | | | | | |

### Example 2: IFN-I Signaling Increased CAR T-Cell Effector Functions

Previous studies have demonstrated the immunomodulatory role of T cell-intrinsic IFN-I signaling (Crouse et al., 2015, Nat. Rev. Immnol. 15, 231-242). For instance, IFN-I signaling promotes cytotoxicity (Curtsinger et al., 2005, J. Immnol. 174, 4465-4469), Th1 polarization (Cho et al., 1996, J. Immnol. 157, 4781-4789; Huber et al., 2010, J. Immunol. 185, 813-817; Ramgolam et al., 2009, J. Immunol. 183, 5418-5427), and central memory phenotype differentiation (Ramos et al., 2009, Blood 113, 5516-5525), all of which may be desired CAR T-cell characteristics and potentially explain the differential clinical responses observed in the two patient cohorts. Based on these findings and the bioinformatics analyses, it was hypothesized that IFN-I signaling can enhance CAR T-cell efficacy. To test this hypothesis, both CD28- and 4-1BB-costimulated CD19-directed CAR T cells were generated using primary T cells from healthy human donors, based on axicabtagene ciloleucel (axi-cel, FIG. 2A) and lisocabtagene maraleucel (liso-cel, FIG. 2B) respectively. To induce IFN-I signaling, CAR T cells were treated with exogenous IFN-α2, a highly potent, FDA-approved, and clinically compatible IFN-I subtype, for culturing CAR T cells (FIG. 2C). Prior to addition of IFN-α2, it was confirmed that CAR T cells globally expressed the type I interferon receptor in a transduction-independent and costimulation-independent manner (FIG. 2D and FIG. 2E).

Since each downstream target of IFN-I signaling exhibits varying sensitivity to IFN-I concentrations (Mendoza et al., 2017, Immunity 46, 379-392), it was hypothesized that the IFN-I culturing concentration may affect CAR T-cell functions. To determine the IFN-I concentrations for this study, IFN-α2 was initially titrated on both CD28- and 4-1BB-costimulated CAR T cells and intracellular interferon regulatory factor 7 (IRF7), a key ISG (Marie et al., 1998, EMBO J. 17, 6660-6669), was measured (FIG. 2F and FIG. 2G). IFN-I signaling reached saturation at concentrations above 3000 IU/mL of IFN-α2, consistent with previous studies (Curtsinger et al., 2005, J. Immnol. 174, 4465-4469; Ramgolam et al., 2009, J. Immunol. 183, 5418-5427). A half-maximal IRF7 upregulation was also observed at approximately 100-300 IU/mL of IFN-α2. Consequently, we established three culturing conditions: IFN-α2 at saturating concentration (“IFNα-hi”, 3000 IU/mL), IFN-α2 near half-maximal effective concentration (“IFNα-lo”, 300 IU/mL), and no IFN-α2 (“IFNα-neg”, 0 IU/mL). It was confirmed that these conditions resulted in concentration-dependent upregulation of ISGs, including MX1, IRF7, and ISG15 (FIG. 2H and FIG. 2I).

CAR T cells were treated based on the three culturing conditions for downstream experimentation (FIG. 2C). Briefly, T cells were transduced with the CAR construct one day after activation (day 1). IFN-I treatment was administered from day 2 to day 7, after which IFN-I was removed. Cells were subsequently expanded until day 13 for experiments. Since IFN-I signaling can inhibit viral transduction (Durgin et al., 2023, Blood 142, 6830), IFN-α2 was added after CAR transduction, which enabled a consistent transduction efficiency and expression of both CD28-costimulated (FIG. 2J and FIG. 2L) and 4-1BB-costimulated CARs (FIG. 2K and FIG. 2M) across the three culturing conditions. IFN-α2 was also removed on day 7 of ex vivo expansion to prevent residual cytokine from inducing potential in vivo toxicity (Quesada et al., 1986, J. Clin. Oncol. 4, 234-243; Trinchieri et al., 2010, J Exp. Med. 207, 2053-2063). Between culturing conditions, IFN-I signaling did not affect CD4/CD8 ratios (FIG. 2N and FIG. 2O), memory phenotypes (FIG. 2P-2S), or IFN-I receptor expression (FIG. 2T-2W). Although long-term sustained IFN-I signaling drives T cell exhaustion in tumor microenvironment (Lukhele et al., 2022, Immunity 55, 2369-2385.e10), the short-term IFN-I exposure in ex vivo culture did not induce exhaustion (FIG. 2X and FIG. 2Y). To test cytotoxicity, CAR T cells were co-cultured with OCI-Ly8 target cells, a CD19⁺ human DLBCL cell line. In both CD28- and 4-1BB-costimulated CAR T cells, IFN-I signaling significantly increased CAR T-cell cytotoxicity (FIG. 2Z and FIG. 2AA), expression of cytolytic molecules (granzyme B, perforin, FIG. 2AB and FIG. 2AC), and secretion of cytokines (IL-2, TNF, IFNγ, FIG. 2AD and FIG. 2AE), in a concentration-dependent manner. These findings suggest that IFN-I signaling increased CAR T-cell cytotoxicity.

### Example 3: High-Strength IFN-I Signaling Increased Apoptosis, Whereas Low-Strength IFN-I Signaling Did not

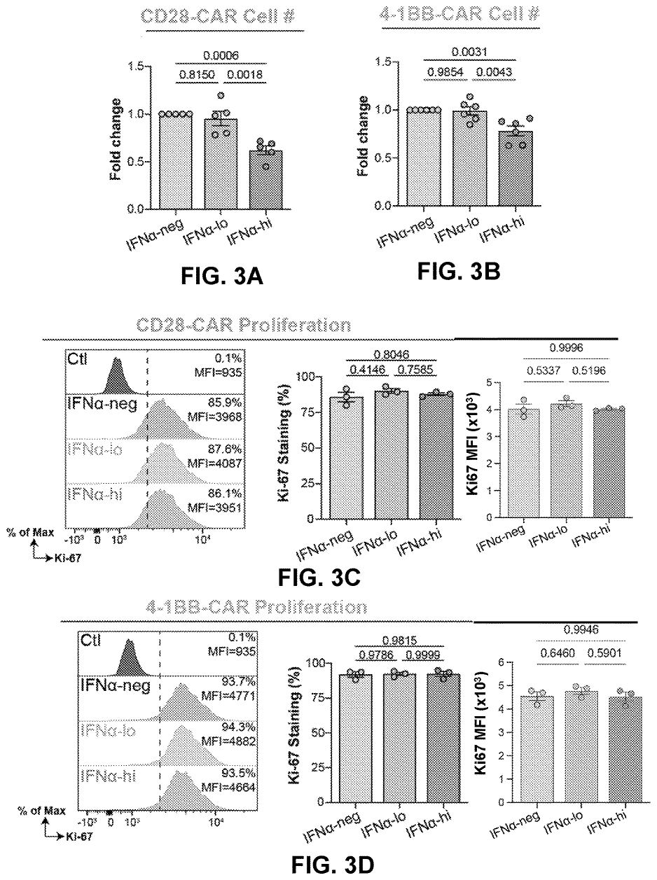

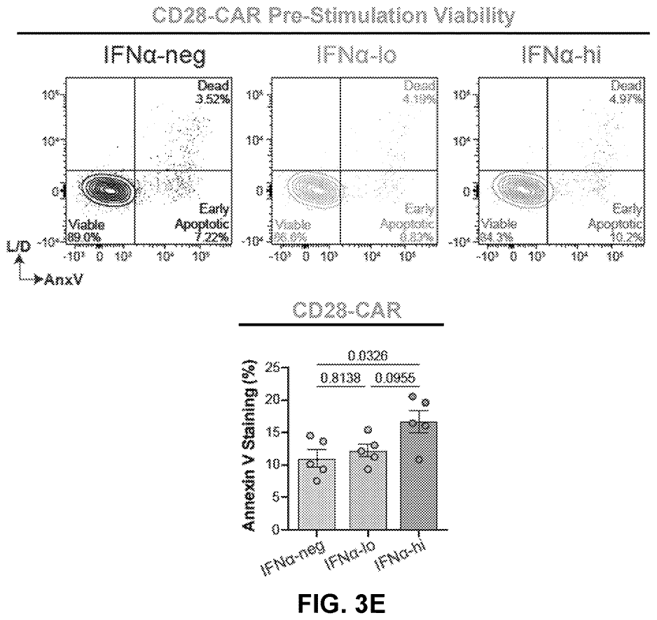

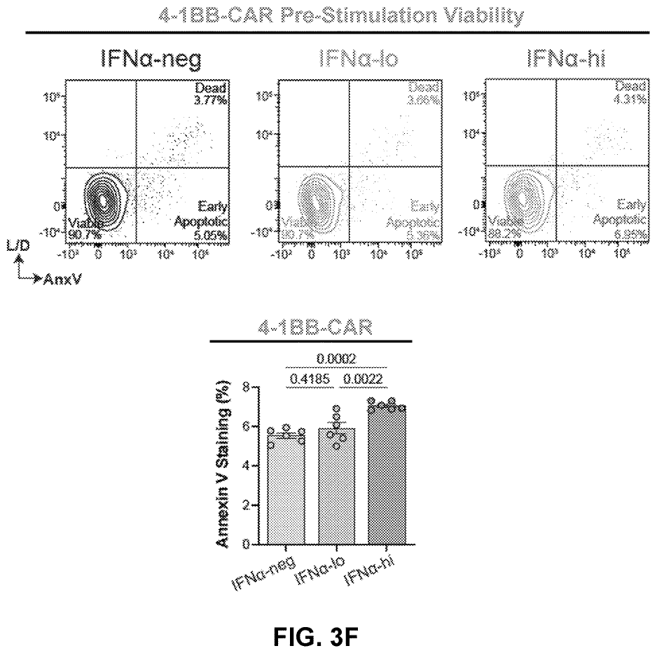

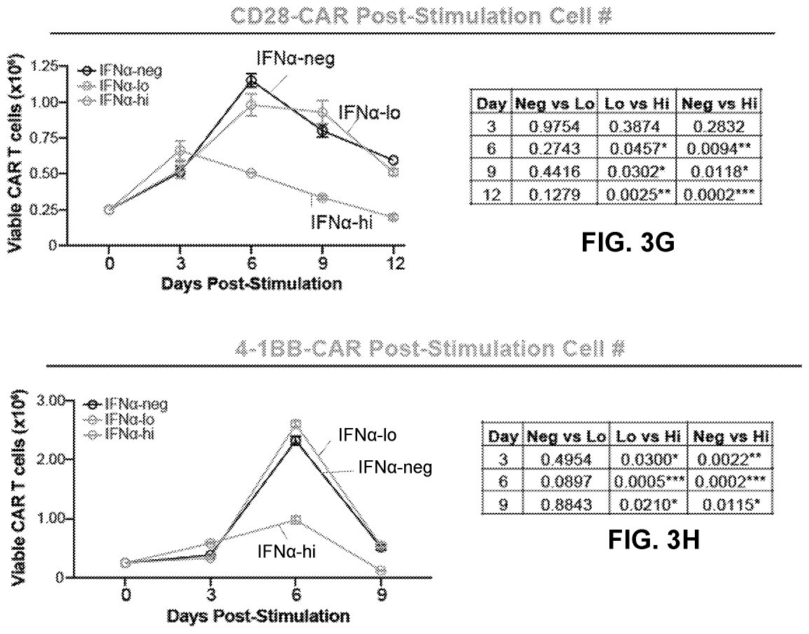

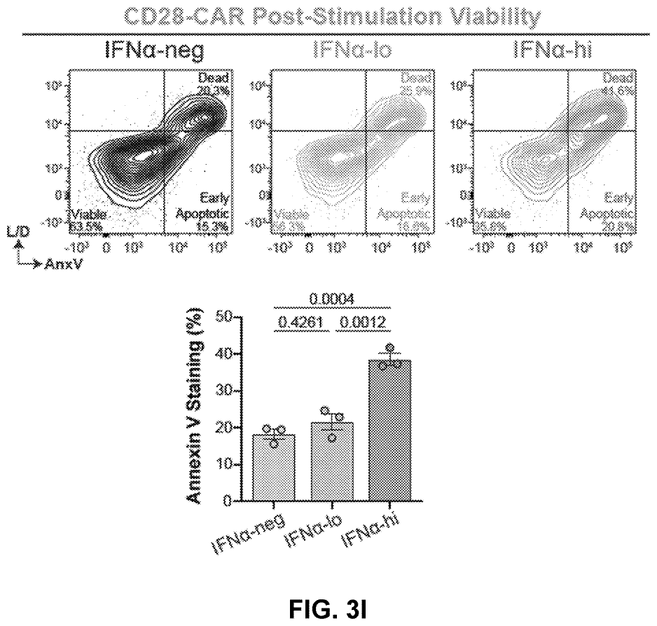

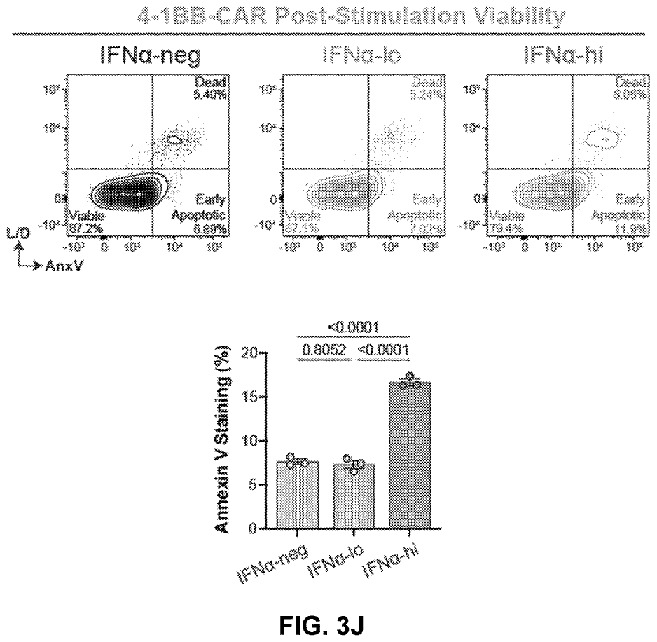

The IFNα-lo condition consistently yielded comparable levels of viable CAR T cells to IFNα-neg controls following manufacturing, whereas the IFNα-hi condition reduced CAR T-cell yield (FIG. 3A, FIG. 3B, and FIG. 3C). Given previous reports that IFN-I signaling can inhibit cell proliferation (Cheon et al., 2023, Trends in Cancer 9, 83-92), experiments were carried out Ele valuate whether CAR T-cell proliferation was affected by IFN-I signaling through assessing the proliferation marker, Ki-67. No significant differences in Ki-67 expression were observed across the three culturing conditions, indicating that IFN-I signaling does not impair CAR T-cell proliferation during ex vivo expansion (FIG. 3C and FIG. 3D). Since IFN-I signaling also reportedly promotes T-cell apoptosis (Harrer et al., 2022, Cells 11, 3839; Evgin et al., 2020, Nat. Commun. 11, 1-15), CAR T-cell apoptosis was quantified via Annexin V staining. Flow phenotyping revealed that CAR T cells in the IFNα-hi condition trended towards increased apoptosis, whereas those in the IFNα-lo condition did not (FIG. 3E and FIG. 3F).

To further investigate the cellularity and apoptosis of CAR T cells from the three culturing conditions following antigen-specific stimulation, cellularity and apoptosis of CAR T cells were subsequently quantified after stimulation with OCI-Ly8 target cells. Following stimulation, CAR T-cell cellularity from the IFNα-lo and IFNα-neg conditions was statistically indistinguishable (FIG. 3G and FIG. 311). On the other hand, CAR T-cell cellularity from the IFNα-hi condition was significantly depleted compared to the other two conditions. At the timepoint when cellularity decline became apparent (day 6 post-stimulation in the representative data), a corresponding enrichment in apoptosis in CAR T cells was observed from the IFNα-hi condition, but not in those from the IFNα-lo condition (FIG. 3I and FIG. 3J).

Taken together, these findings suggest that high-strength, but not low-strength IFN-I signaling increases CAR T-cell apoptosis, thereby negatively affecting persistence during both ex vivo expansion and antigen-specific activation

### Example 4: Low-Strength IFN-I Signaling Increased Cytotoxicity of DLBCL Patient-Derived CAR T Cells

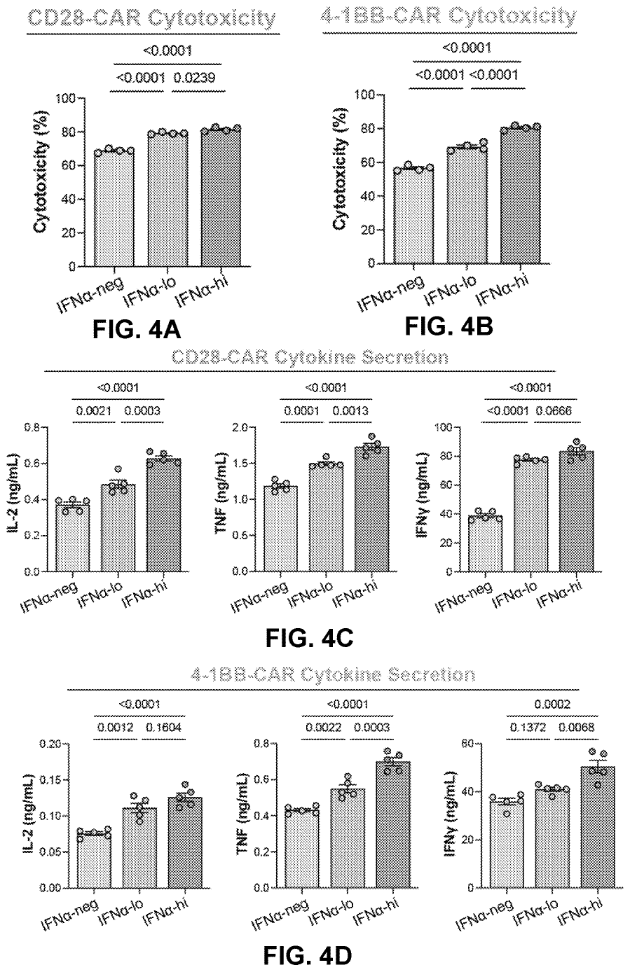

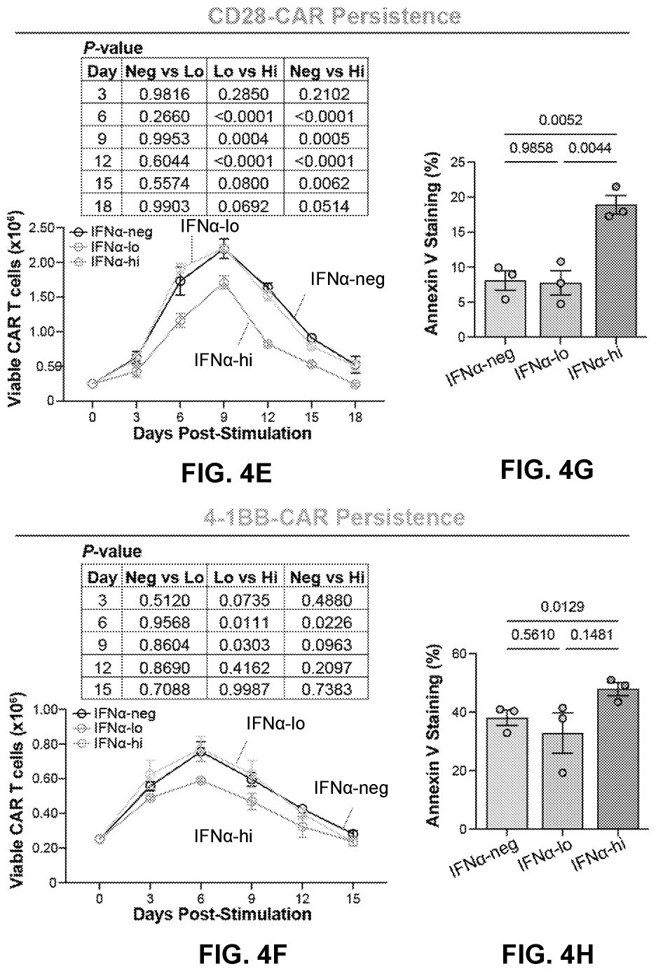

To evaluate the clinical translation potential of our IFN-I enhancement process, IFN-α2 was tested with CAR T cells generated from the leukapheresis product of a patient with DLBCL. IFN-I signaling increased CAR T-cell cytotoxicity (FIG. 4A and FIG. 4B) and cytokine secretion (IL-2, TNF, IFNγ; FIG. 4C and FIG. 4D) in a dose-dependent manner. Upon stimulation with OCI-Ly8 target cells, CAR T cells from the IFNα-hi condition demonstrated decreased persistence and increased apoptosis, whereas those from the IFNα-lo condition maintained similar persistence compared to IFNα-neg controls without increased apoptosis (FIGS. 4E-4H). Findings were consistent across both CD28- and 4-1BB-costimulated CAR constructs (FIGS. 4A, 4C, 4E, and 4G compared to FIGS. 4B, 4D, 4F, and 411). These findings demonstrate that low-strength IFN-I signaling enhances effector function without compromising apoptosis and persistence for all primary human T cells, including those from patients. These conclusions significantly augment the clinical translatability of the findings.

### Example 5: Low-Strength IFN-I Signaling Promoted CAR T-Cell Treatment Efficacy In Vivo

Given that the IFNα-lo condition uniquely increased CAR T-cell functionality (FIGS. 2A-2AE) without compromising cell viability (FIGS. 3A-3J and FIGS. 4A-4H) in both CD28- and 4-1BB-costimulated CAR T cells, the data demonstrate that low-strength IFN-I signaling promoted CAR T-cell efficacy in vitro by balancing function with persistence. Based on these in vitro conclusions, it was hypothesized that the IFNα-lo condition uniquely optimizes CAR T-cell treatment efficacy in vivo.

To test the hypothesis, the efficacy of CAR T cells prepared from the three culturing conditions in a xenograft mouse model based on the OCI-Ly8 cell line was analyzed (FIG. 5A). To control for donor-donor heterogeneity, CAR T cells were derived from multiple independent donors (three for CD28-costimulated CAR T cells and two for 4-1BB-costimulated CAR T cells). Across donors, CAR T-cell injection prolonged survival relative to the PBS-only control. For CD28-costimulated CAR T cells with donors 1 and 2, CAR T cells from the IFNα-lo condition significantly prolonged survival and slowed down tumor progression relative to CAR T cells from the IFNα-neg and IFNα-hi conditions (FIG. 5B and FIG. 5G). For donor 3, although the differences between CAR T cells from IFNα-lo and IFN α-hi conditions were relatively modest compared to other donors, one mouse treated with CAR-T cells from the IFNα-lo condition showed a significantly better and sustained response (FIG. 5D and FIG. 511). For 4-1BB-costimulated CAR T cells with donors 4 and 5, CAR T cells from the IFNα-lo condition consistently yielded superior outcomes compared to CAR T cells from the IFNα-neg and IFNα-hi conditions, leading to improved survival and tumor control (FIG. 5C and FIG. 5I). Taken together, these findings support the hypothesis and indicate that CAR T cells generated from the IFNα-lo condition, regardless of costimulatory domain, exhibited enhanced in vivo efficacy compared to those from IFNα-neg or IFNα-hi conditions across multiple donors.

### Example 6: Low-Strength IFN-I Signaling Promotes CAR T-Cell Efficacy in Other CD19⁺ Tumor Model

It was hypothesized that low-strength IFN-I signaling could also enhance CAR T-cell efficacy against other CD19⁺ tumors. To test this, the cytotoxicity of CAR T cells generated under three culturing conditions were tested against two additional CD19⁺ tumor cell lines: VAL (a DLBCL cell line) and NALM6 (a leukemia cell line). Consistently, IFN-I treatment enhanced CAR T-cell cytotoxicity for both costimulatory CAR constructs in a dose-dependent manner (FIGS. 6A-6D). Moreover, low-strength IFN-I signaling did not increase apoptosis or compromise CAR T-cell persistence, whereas high-strength IFN-I signaling did (FIGS. 6E-6L). In the NALM6-based xenograft in vivo model, both CD28- and 4-1BB-costimulated CAR T cells generated under IFNα-lo conditions outperformed the other conditions, as evidenced by prolonged survival and reduced tumor burden (FIGS. 6M-6Q). Together, these data suggest that the IFNα-lo approach had broad applicability for enhancing CAR T-cell efficacy against other CD19⁺ tumors.

### Example 7: Low-Strength IFN-I Signaling Promoted Durable CAR T-Cell Efficacy

Experiments were set up to further assess whether CAR T cells generated under IFNα-lo conditions could promote durable efficacy by modifying the original OCI-Ly8 model to include an additional tumor challenge following initial tumor control after CAR T-cell treatment (FIG. 7A). CAR T cells generated under IFNα-lo conditions conferred superior survival benefit and tumor control compared with those generated under IFNα-neg conditions (FIG. 7B and FIG. 7C). This was accompanied by higher levels of CAR T cells in the peripheral blood and spleen at later time points following the second challenge (FIG. 7E and FIG. 7F), although memory phenotypes were not markedly altered (FIG. 7G and FIG. 711). Collectively, these data suggest that the IFNα-lo approach promoted durable CAR T-cell efficacy.

In conclusion, the in vitro and in vivo studies presented herein support a model in which low-strength IFN-I signaling selectively promoted CAR T-cell treatment efficacy (FIG. 7G). IFN-I signaling promoted CAR T-cell functionality in a concentration-dependent manner, leading to more effective cytotoxicity, cytokine release, and tumor control. However, at a high enough concentration, IFN-I signaling also increased T-cell apoptosis, leading to reduced yield, poorer persistence, and ineffective tumor control. Although the optimal level of IFN-I signaling may be subject to donor-donor heterogeneity, CAR T cells treated with low-strength IFN-I signaling, regardless of costimulatory domain, consistently exhibit enhanced effector functions without significantly increased apoptosis.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

What is claimed is:

1. A population of IFN-enhanced CAR-T cells having about 40% to about 70% maximal expression of one or more interferon-stimulated genes induced by treatment with an IFN receptor agonist.

2. The population of IFN-enhanced CAR-T cells of claim 1, wherein the interferon-stimulated gene is one or more of OAS1/2/3, OASL, ADAR, IFITM1/2/3, IFI6, IFI16, ZAP, APOBEC3, ISG20, SOCS1, IRF7, MX1, and ISG15.

3. The population of IFN-enhanced CAR-T cells of claim 1, wherein the interferon-stimulated gene is IRF7.

4. The population of IFN-enhanced CAR-T cells of claim 1, wherein the IFN-enhanced CAR-T cells bind to CD19, CD20, CD22, or BCMA.

5. The population of IFN-enhanced CAR-T cells of claim 3, wherein the IFN receptor agonist is an IFN type I (IFN-I) receptor agonist, an IFN type III (IFN-III) receptor agonist, or a combination thereof.

6. The population of IFN-enhanced CAR-T cells of claim 5, wherein the IFN-I receptor agonist is IFN-α2.

7. The population of IFN-enhanced CAR-T cells of claim 6, wherein the treatment comprises exposure of a population of CAR-T cells to a concentration of IFN-α2 between about 100 IU/mL and about 1000 IU/mL.

8. The population of IFN-enhanced CAR-T cells of claim 7, wherein the concentration of IFN-α2 is about 300 IU/mL.

9. The population of IFN-enhanced CAR-T cells of claim 8, wherein the IFN-enhanced CAR-T cells express a CAR gene construct that is an axicabtagene ciloleucel construct or a lisocabtagene maraleucel construct.

10. The population of IFN-enhanced CAR-T cells of claim 1, wherein the population of IFN-enhanced CAR-T cells:
a) produces more IL-2, TNF, and/or IFNγ upon stimulation compared to a control population of CAR-T cells that is produced without treatment with the IFN receptor agonist;
b) expresses a higher level of interferon-stimulated genes compared to a control population of CAR-T cells that is produced without treatment with the IFN receptor agonist;
c) has the same rate of T cell activation-induced apoptosis as a control population of CAR-T cells that is produced without treatment with the IFN receptor agonist;
d) is more effective in killing tumor cells compared to a control population of CAR-T cells that is produced without treatment with the IFN receptor agonist; and/or
e) has more durable efficacy compared to a control population of CAR-T cells that is produced without treatment with the IFN receptor agonist.

11. A method of producing a population of interferon (IFN)-enhanced chimeric antigen receptor (CAR)-T cells, comprising:
a) introducing a CAR gene construct into a population of T cells to produce a population of CAR-T cells; and
b) co-culturing the population of CAR-T cells with a concentration of an IFN receptor agonist to obtain a population of IFN-enhanced CAR-T cells that exhibits about 40% to about 70% maximal expression of an interferon-stimulated gene.

12. The method of claim 11 further comprising activating T cell receptor (TCR) signaling in the population of T cells or in the population of CAR-T cells before step (b).

13. The method of claim 12, wherein the TCR signaling is activated by CD3 and/or CD28.

14. The method of claim 11 further comprising step (c) expanding the population of IFN-enhanced CAR-T cells.

15. The population of IFN-enhanced CAR-T cells of claim 11, wherein the interferon-stimulated gene is one or more of OAS1/2/3, OASL, ADAR, IFITM1/2/3, IFI6, IFI16, ZAP, APOBEC3, ISG20, SOCS1, IRF7, MX1, and ISG15.

16. The population of IFN-enhanced CAR-T cells of claim 15, wherein the interferon-stimulated gene is IRF7.

17. The method of claim 16, wherein the CAR gene construct encodes a CAR that binds to CD19, CD20, CD22, or BCMA.

18. The method of claim 16, wherein the IFN receptor agonist is an IFN type I (IFN-I) receptor agonist, an IFN type III (IFN-III) receptor agonist, or a combination thereof.

19. The method of claim 18, wherein the IFN-I receptor agonist is IFN-α2.

20. The method of claim 19, wherein the concentration of the IFN-α2 is between about 100 IU/mL and about 1000 IU/mL.

21. The method of claim 20, wherein the concentration of the IFN-α2 is about 300 IU/mL.

22. The method of claim 21, wherein the CAR gene construct is an axicabtagene ciloleucel construct or a lisocabtagene maraleucel construct.

23. The method of claim 11, wherein the population of IFN enhanced CAR-T cells:
a) produces more IL-2, TNF, and/or IFNγ upon stimulation compared to a control population of CAR-T cells that is produced without co-culturing with the IFN receptor agonist;
b) expresses a higher level of interferon-stimulated genes compared to a control population of CAR-T cells that is produced without co-culturing with the IFN receptor agonist;
c) has the same rate of T cell activation-induced apoptosis as a control population of CAR-T cells that is produced without co-culturing with the IFN receptor agonist;
d) is more effective in killing tumor cells compared to a control population of CAR-T cells that is produced without co-culturing with the IFN receptor agonist; and/or
e) has more durable efficacy compared to a control population of CAR-T cells that is produced without co-culturing with the IFN receptor agonist.

24. A population of IFN-enhanced CAR-T cells produced by the method according to claim 11.

25. A pharmaceutical composition comprising the population of IFN-enhanced CAR-T cells of claim 24 and a pharmaceutically acceptable carrier.

26. A method of treating cancer in a patient, comprising administering a therapeutically effective amount of the pharmaceutical composition of claim 25 to the patient.

27. The method of claim 26, wherein the cancer is leukemia, multiple myeloma, diffuse large B cell lymphoma, acute myeloid leukemia, Mantle Cell lymphoma, follicular lymphoma, primary mediastinal B-cell lymphoma, acute lymphoblastic leukemia, or chronic lymphocytic leukemia.

28. The method of claim 27, wherein the cancer comprises cancer cells that express CD19.

29. The method of claim 26, wherein overall survival of the subject is increased by at least about 2 days.

30. The method of claim 29 further comprising increasing progression-free survival, increasing radiographic response, and/or increasing patient quality of life.

31. A cell culture, comprising:
a) a population of CAR-T cells; and
b) a culture medium comprising a concentration of an IFN receptor agonist sufficient to increase expression of an interferon-stimulated gene in the population of CAR-T cells to about 40% to about 70% of maximal expression.
